(19) United States
(12) Patent Application Publication
(10) Pub. No.: US 2025/0246259 A1
(43) Pub. Date: Jul. 31, 2025

(54) METHOD FOR PREDICTIVE TESTING OF AGENTS TO ASSESS TRIGGERING AND SUPPRESSING ADVERSE OUTCOMES AND/OR DISEASES

(71) Applicant: INFINITE d.o.o., Limbus (SI)

(72) Inventors: Janez Strancar, Limbus (SI); Iztok Urbancic, Limbus (SI)

(21) Appl. No.: 18/853,117

(22) PCT Filed: Mar. 30, 2022
(86) PCT No.: PCT/EP2022/058512

§ 371 Date: Sep. 30, 2024

(51) Int. Cl. G16B 5/00

(52) U.S. Cl. CPC G16B 5/00

(57) ABSTRACT

A method for predictive testing of materials allows saving time in observation of adverse outcomes such as chronic inflammation, which is known to lead to development of most of the slowly evolving diseases such as cancer or autoimmune diseases. Observation of evolution of in vitro models may be used to predict outcomes based on early molecular events that are the first to occur, long before they are shown in tissues as long-term effects. These molecular events are analysed, measured and/or detected for up to 1 week and in silico time propagation is performed, in order to correlate the early events to the long-term effects leading a reliable prediction about safety/toxicity of materials.

### CROSS REFERENCE TO RELATED APPLICATION

This present application is a § 371 national stage of International Application PCT/EP2022/083516, filed Mar. 30, 2022, which is hereby incorporated herein by reference in its entirety.

### FIELD

The present disclosure belongs to the field of investigating or analysing materials, especially by determining their biological effect due to their chemical or physical or biological properties. The disclosure also belongs to the field of safety testing by determining biological events as well as to field of computing and calculating those effects further in time. The disclosure relates to a method for predictive testing of agents, such as toxicants, materials, chemicals, medicines and vaccines to assess triggering and suppressing adverse outcomes and/or diseases.

### BACKGROUND

Substances (agents), such as materials and particulate matter, compounds, drugs or candidate medications, and vaccines have to be tested in order to detect any possible toxicity, adverse outcomes or similar unwanted scenarios, among which the slowly evolving adverse outcomes are particularly problematic. Currently, animal testing is the only method able to detect long-term (i.e. chronic) health complications of substances that do not show immediate toxicity neither strong acute inflammatory response. Namely, animals are exposed and monitored for months to detect the long-term effect of substances-materials or compounds. The testing may be research driven, focusing on developing fundamental mechanistic knowledge of an organism, or applied to answer some questions of great practical importance, such as testing disease treatments, breeding, defence research and toxicology, including material, drug and vaccine safety and cosmetics testing. It is estimated that the annual use of vertebrate animals, from zebrafish to non-human primates, exceeds 100 million. Mice, rats, fish, amphibians and reptiles together account for over 85% of research animals.

Animal testing, however, has several problems. Because it is focused on detection of end-points (adverse outcome), it is slow and thus expensive, often unethical, and sometimes even irrelevant by failing to accurately mirror outcomes in humans in some cases. For instance, Greek and Menache (2013, doi:10.7150/ijms.5529) noted that some 100 vaccines have been shown to prevent HIV in animals, yet none of them have worked on humans. On the contrary, Braken (2009, doi:10.1258/jrsm.2008.08k033) showed that thalidomide causes serious birth defects in humans, but not in animals. Nevertheless, the main obstacle in broad application of material and chemical safety testing is undoubtedly long time to reach end-point of long-term or chronic diseases. The required time scale of months makes such testing absolutely useless in case of material and chemicals safety testing, where much faster decisions are required during development processes.

Thus, replacement of animals, i.e., alternatives to animal testing, is clearly needed. Official bodies such as the European Centre for the Validation of Alternative Test Methods (ECVAM) of the European Commission (EC), the Interagency Coordinating Committee for the Validation of Alternative Methods in the US, ZEBET in Germany, and the Japanese Center for the Validation of Alternative Methods, promote abolishment of animal testing and respond to regulatory requirements.

These needs are the driving force behind development of various testing platforms that are all upgraded cell cultures systems. The most logical way seems to be development of structural copies of tissues, on which testing is performed. Here, the rationale behind is structural similarity between the original tissue and a copy such as organoids (organ-like testing devices), “organs-on-a-chip” (patent applications US2020408744, WO2020172670 and WO2009146911), “lung-on-a-chip” (patent applications GB2585150 and WO2018217882), “gut-on-a-chip” (patent applications US2020224136; US2020283732), “skin-on-a-chip” (patent application US2020239857). The systems contain cells in a 2- or 3-dimensional system that mimics organs. These chips can be used instead of animals in in vitro disease research, drug testing, and toxicity testing. Usually, such chips are intended for use in gene expression studies (patent applications US2020224136, WO2020172670), sometimes paired with cell morphology screening and determination of specific enzyme activity or metabolic event (patent applications WO2020172670, JPH04148695, EP2154241, WO2007120699). For, example Wyss Institute (https://wyss.harvard.edu/technology/using-systems-biology-to-find-and-test-new-drugs-faster/) reports on a novel algorithm used for drug discovery, wherein the algorithm is arranged to sift through the gene expression pattern data of tens of thousands of known drug compounds and to identify those that have the potential to revert a disease-state expression pattern to a normal one. These candidate compounds should then be tested further in cell-based assays to evaluate in vitro activity. In the last step, successfully compounds are tested both in cell-based assays and in human organ chips to validate that they can effectively treat the disease without harmful side effects before they move on to clinical trials. Although representing a good alternative, the time to test possible adverse outcomes on such organ-on-chip systems remains to be similar to the in vivo animal testing (for example, patent application WO2020172670 describes methods of testing kidney chips that lasts for more than a week, some of the methods taking up four weeks of exposure), which consequently means that such testing is still expensive and long lasting.

Another alternative is in silico or computer simulation and mathematical modelling which seeks to investigate and ultimately predict toxicity and drug affects in humans without using animals, tissues or cells (Watts and Geoff, 2007, doi:10.1136/bmj.39058.469491.68; Edelman et al, 2010, doi:10.1002/wsbm.75. PMC 3157287. PMID 20836040). This is achieved by investigating test compounds on a molecular level using recent advances in computational capabilities with the ultimate goal of creating treatments unique to each patient. The most recent development of In silico testing methods are given in the recent review (Mark T. D et al. Computational Toxicology 21, 2022, https://doi.org/10.1016/j.comtox.2022.100213) where predictions of hazard and exposure relies on the QSARs and read-across approaches. The main problem of this approach is the lack of biological testing, as modelling may not take into account specific or more complex cell strategies that are initiated upon exposure to a potential toxic compound. Such modelling can rely only on the already available information and knowledge (for example on AOPs) or derivable from molecular dynamics simulations, which are currently capable of simulating the response of only a small volume of cellular content. Because in silico simulations have to be performed on super computers, they are therefore also costly and time-consuming.

Having the above-mentioned in mind, an efficient, reliable, fast and cost-efficient method for testing different materials and compounds is needed that will avoid use of animals or isolated in silico modelling.

Although many methods have been proposed and developed as alternatives for animal testing, which were partly mentioned above, this section includes only exemplary methods with the aim to emphasize the novelty of the approach used in the present disclosure.

Patent application WO0047761 discloses a method for analysing toxicity of a chemical, wherein a wildtype and a mutant strain of the same cell type, i.e., yeast cell, bacterial cell, cell-line of human origin, are used to detect oxidative stress, protein damage, cell cycle disruption, energy charge and depletion, microtubule disruption or onset of metabolic competency through overexpression of human gene inserts encoding metabolism genes or incorporation of S9 fraction. In one embodiment of the present disclosure, wildtype yeast and respective mutants are dosed with the desired chemical and yeast growth is determined using turbidimetry. Dose response curves are generated and mutant sensitivity to the compound relative to its parent (relative sensitivity) calculated. Relative sensitivities which are statistically significant indicate a hypersensitivity of the mutant to the test compound. This approach differs significantly from the present disclosure, as there is no need for mutant strains or calculation of relative sensitivities.

Patent application WO2009146911 describes a self-contained sensor-controlled organ-on-a-chip device, which allows establishing or maintaining organs or organoids as well as stem cell niches in a miniaturized chip format, suitable for online observation by live cell imaging and for example two photon microscopy. Use of the organ-on-a-chip device in testing activity, pharmacodynamic and pharmacokinetic of compounds is also described, wherein the testing method comprises the following steps:
Providing the organ-on-a-chip device,
Adding one or more test compounds to the organ/organoid on the chip,
Assessing the organ/organoid microscopically and/or determining one or more parameter determinable by one or more sensors.

The document is, however, silent about the particulars of the testing method, the parameters or expected microscopic changes in the organ/organoid. Despite this, it is evident that only one time point after exposure is taken to observe possible effects of test compounds on the organ/organoid without any profound analysis of the impact of this effect on long-term effects.

Murschhauser et al (Communications Biology volume 2, Article number: 35; 2019) describe a high-throughput microscopy method for single-cell analysis of event-time correlations in nanoparticle-induced cell death. Particularly, the method is applied to extract event times from fluorescence time traces of cell death-related markers in automated live-cell imaging on single-cell arrays (LISCA) using epithelial A549 lung and Huh7 liver cancer cells as a model system. In pairwise marker combinations, the chronological sequence and delay times of lysosomal membrane permeabilization, mitochondrial outer membrane permeabilization and oxidative burst after exposure to 58 nm amino-functionalized polystyrene nanoparticles (PS-NH2 nanoparticles) were assessed. Results indicate that different cells at different doses of nanoparticles show heterogeneity and interdependencies in signal transmission pathways. This solution does not offer any prediction about the possible adverse effects of nanoparticles, but rather shows mechanisms of signalling pathways and cellular responses to various doses of nanoparticles. Although the authors anticipate that the high-throughput assay will become a powerful tool for the study of mechanistic and temporal heterogeneity in cellular responses to diverse types of perturbation, they do not show how this can translate into adverse-outcome prediction yet alone perform such a prediction.

To sum up, all currently known methods of material testing and toxicity evaluation are based on measurement or detection of a group of specific events in cells and/or tissues and/or organ-on-chip assemblies after exposure. In contrast, the present disclosure aims to predict long-term toxicity based on an early cellular event using time propagation. It thus upgrades early event monitoring by additional construction of a mathematical model, that automatically learns about early mechanisms and propagates the observed early events evolutions into late adverse outcome prediction.

Patent application WO2018217882 discloses a microfluidic Small Airway-on-Chip that was infected with one or more infectious agents (e.g., respiratory viruses) as a model of respiratory disease exacerbation (for example, asthma exacerbation). Upon exposure, the cells of the lung epithelium in the chip were analysed for expression of phenotypes characteristic for asthma. Various phenotypes were observed, but their recognition was based on knowledge of asthma exacerbation mechanisms. Although that disclosure represents a complete approach to observation of toxicant-induced early molecular changes in advanced in vitro system, it does not predict how these events evolve in time, which is addressed by the methods of the present disclosure. Thus, it does not replace end-point testing, it only monitors the early evolution.

If one would know the mechanism of event propagation in advance and would be able to automatically translate in vitro models monitoring into mathematical description of the events' propagation, then the time propagation could be done by the model suggested recently by Kokot et al. (2020, doi:10.1002/adma.202003913). In this work, the authors have first explored the toxicity of inhaled nanomaterials and reported on a discovery of new molecular key events and their causal relationships, wherein the events were the process of nanomaterial quarantining (nano-quarantining) and nanomaterial cycling between different lung cell types, which led the cells into a vicious circle of chronic inflammation. A similar cycling events (loops) have been observed in Covid-19 pneumonia cases as reported by Mould and Janssen (2021, Nature, Vol 590, 553).

After the mechanisms have been discovered, a chain of causally related events was described by a simplified theoretical model that describes the flow of the nanomaterial between four separate compartments: i) outside the cells, ii) inside the epithelial cells, iii) quarantined in cauliflowers, and iv) inside macrophages. This model is defined via several parameters, which can however be simplified into three key descriptors:
a) The rate of toxicity of the nanomaterials to individual cells (determined by the measured number of viable macrophages in a MH-S monoculture after 4 days of exposure);
b) The rate of nanomaterial quarantining by epithelial cells (calculated from the measured fraction of nanomaterial in the cauliflowers in the LA-4 monoculture after 2 days of exposure) taking into account the correction due to the rate of toxicity of the nanomaterials to individual cells;
c) The efficiency of the signalling and the monocyte influx replacing the dying macrophages (calculated from the measured influx of inflammatory cells (leukocyte) in vivo after at least 10 days), a time point where the development of chronic events of the response is started; the calculation includes the corrections due to the rate of toxicity of the nanomaterials to individual cells (as well as due to the rate of nanomaterial quarantining by epithelial cells.

Based on this mechanistic understanding and the corresponding mathematical formulation including simplified parameterization, the authors attempted prediction of nanomaterial-induced chronic inflammation through these nanomaterial descriptors determined from three single time-point measurements: two in vitro and one in vivo, all measured few days after the exposure. Using the above-mentioned rates, the model was able to reproduce the in vivo time course of the amount of quarantined nanomaterial in the cauliflowers, signalling for immune cells influx, as well as of the total macrophage number, which can be used to predict the nanomaterial-specific acute-to-chronic inflammation outcome. The paper is, however, silent about how the time courses of various in vitro and in vivo observable events are translated into prediction of adverse outcomes itself. Moreover, the screening strategy in material safety assessment proposed by Kokot et al. is based on understanding of the response of the organism to nanomaterial exposure from the initial contact with the nanomaterial to the potential adverse outcome. This means that a known molecular mechanism is needed to predict toxicity of tested materials as proposed, ultimately requiring multidisciplinary approaches, possibly combining advanced imaging, omics, particle labelling, and tracking techniques applied in vivo and in vitro with in silico modelling. Consequently, for each material significant effort should be channelled into discovery of mechanisms of effects that materials have on a specific type of cell, which undoubtedly leads to long and expensive toxicity testing.

A disadvantage of the solution according to Kokot et al. and other cited documents is that for each new material, a mechanism (mode of action) must first be discovered before new observables and their interaction terms can be introduced into the model for prediction of material toxicity. Furthermore, known methods based on in vitro and in vivo experiments are performed in a destructive manner delivering only information about events in one time-point only.

### SUMMARY

The present disclosure aims to provide an upgraded method for predictive testing of materials, chemicals, medicines, vaccines and similar compounds in order to assess their safety, possible adverse outcomes and/or triggering of diseases. The present disclosure upgrades monitoring of early events with a method, that automatically learns about early mechanisms by translating in vitro observed early events' propagation into a mathematical form that is in turn used to propagate the early events' evolution into late adverse outcome prediction. The technical problem is solved by the method described in the independent claim, while some embodiments of the method are defined in dependent claims.

The main advantage of the disclosed methods is absence of the need to understand mechanisms of tested compounds (mode of action) while translating non-destructive time-lapse monitoring of living in vitro models into mathematical description of evolution of observables (key cellular events) and the latter into prediction of in vivo-observable adverse outcome. The method inherently allows automatic discovery of mode of action of the tested agents, such as materials, chemicals, medicines, vaccines or similar substances or particulate matter on the cells. The method further allows determination of systemic effects of the tested substances. Substances that can be tested with the method according to the disclosure may be selected in the group consisting of materials, nanoparticles, vaccines, compounds, chemicals, medicaments, etc. . . . . It is not intended that the present disclosure be limited by the particular tested agent or by particular cells on which the agents are tested.

In order to better understand the subject-matter, the meaning of the following expressions used throughout the text is explained below.

| | |
|---|---|
| In vivo | Experiments on animals |
| In vitro | Experiment on cellular systems (cell lines, tissue-on-chip, organ- |
| | on-chip, organism-on-chip or organoids and any dependent version |
| | of the afore-mentioned) |
| In silico | Numerical experiments run as simulations/modelling taking into |
| | account existing knowledge and parameterization by finite number |
| | of mathematical equations |
| Events | Change of molecular (for example lipid, protein, RNA, DNA, etc.) |
| | or supramolecular (for example membrane, ribosome, |
| | cytoskeleton, fibers, vesicles, etc.) or cellular (cell surface, volume, |
| | shape, activity, cellular compartment surface, volume, shape, etc.) |
| | property that can be quantified: all kinds of molecular structures, |
| | such as primary, secondary, tertiary, etc, 3D shape, aggregation/ |
| | dissociation, release/absorbance (of a molecule, vesicle, etc from |
| | one inter/intra-cellular compartment into another), transcription of |
| | DNA, RNA, expression of a protein from DNA/RNA, binding state, |
| | charge, interaction surface chemical composition, type of |
| | interaction exhibited by the interaction surface, etc. |
| Adverse | Symptom or groups of symptoms (i.e. adverse outcomes (AO)) |
| outcome | associated with, or preceding the onset of, a disease |
| (AO) | |
| Key event | An event that unavoidably leads to the next event in a chain of |
| (KE) | events from exposure to adverse outcome (AO) |
| Exposure | First contact of an agent, i.e., possible toxicant (chemical, drug, |
| | virus, vaccine, particle, material, etc.) with a living system |
| | (cell or tissue barrier, etc.) |
| Observables | Any event-related property i that can be measured experimentally |
| (qᵢ) | in living system (in vitro or in vivo) in terms of concentration, |
| | surface, amount, size, etc. (q) at certain time point (t); |
| | qᵢ must refer to a quantity that is detectable in vitro and |
| | corresponds to or can be associated with a quantity detectable in/ |
| | ex vivo and associates with candidate for key event (KE) |
| Time-lapse | Measurement of observables q at different time points q(t), from |
| | which the rates of changes of q in time dq/dt can be obtained |
| Calibration | A process of calibrating mathematical model of in vitro model |
| | evolution (terms and parameters) to in vivo values in the time scale |
| | of in vitro experiment(s) by matching the latest event observable |
| | in vitro to the earliest event observable in vivo |
| Validation | A process of validating the coupled in vitro - in silico predicted |
| | adverse outcome against in vivo observed adverse outcome far |
| | beyond the time scale of in vitro experiment(s) |
| Scenario (S) | A group of measurable observables S = {qᵢ} that is associated with |
| | particular experimental setup (combination of in vitro (sub)system |
| | and exposure) |
| Adverse | A chain of events initiated by exposure, followed by the first event, |
| Outcome | i.e. Molecular initiating event (MIE, right after exposure or first |
| Pathway | contact), which leads via a series of causally connected Key |
| (AOP) | events (KE) to the Adverse outcomes/effects (AO) |
| Interaction | Mathematical construct that defines the vector of time derivatives |
| matrix (IM) | of observables [symbol] = {\dot{q}ᵢ} = IM(t) · [symbol] from a vector of observables |
| | [symbol] = {qᵢ} for a given scenario S = {u₁, u₂, . . . }, where uᵢ are unity |
| | values 0 or 1 (and represent whether individual observables qi are |
| | selected in the scenario S) |
| Interaction | Element of the interaction matrix IMᵢⱼ, associated with the time |
| term (IMᵢⱼ) | derivative of the observable qᵢ resembling the interactions |
| | between the chosen observable qᵢ and other observables qⱼ. Each |
| | term comprises of a power parameter pᵢⱼ and at least two |
| | interaction function (IFᵢ, IFⱼ) explained below. |
| Time | In silico forecasting of observables in time from their initial values |
| propagation | [symbol] (t = 0) using the system of differential equations, defined via |
| | scenaria and observables that drive the interaction terms of the |
| | interaction matrix: [symbol] (t) = IM(t) · [symbol] |
| Power | A number pᵢⱼ that defines the relative strength of the interaction term |
| parameter | IMᵢⱼ |
| (pᵢⱼ) | |
| Interaction | Function IF(qᵢ, o₁, o₂) that defines the dependence of the |
| function (IF) | Interaction matrix term IMᵢⱼ on the observable qᵢ; defines its |
| | sensitivity to the small values of qᵢ and large values of qᵢ via |
| | derivatives o₁ and o₂, respectively (e.g. a combination o₁ = 0, o₂ = 1 |
| | denotes that such IM is insensitive to variations for qᵢ << 1 and |
| | linearly depends on qᵢ >> 1). With interaction functions IF(qᵢ), |
| | interaction matrix term IMᵢⱼ can be defined as a products of a |
| | power parameter pᵢⱼ. (strength of the Interaction matrix term) and at |
| | least two interaction functions IF(qₖ), where k can be the index of |
| | the row i or column j, depending on the group of observables/ |
| | position of Interaction matrix terms IMᵢⱼ. |
| Base | Base function Bij is sum of products of interaction functions IF(qₖ), |
| function (Bᵢⱼ) | associated with the power parameter pᵢⱼ, creating base of linearly |
| | independent functions able to describe the time derivative of |
| | individual observable qᵢ; |
| Super | Super equation refers to a subgroup of differential equations |
| equation | (group of rows of Interaction matrix) in which only a subgroup of |
| | power parameters Pa appears with no other power parameters |
| | present (this group being mathematically a complement of the |
| | subgroup parameters Pa). |

The alternative testing method according to the disclosed methods saves time in associating slowly-evolving adverse outcomes with possible triggering agents, which is done by replacing classical observation of late adverse outcomes with monitoring early key events evolution in vitro and further propagation of this evolution in time in silico.

Such a time saving can be easily exemplified on the case of development of chronic inflammation, which is known to further lead to development of cancer or autoimmune diseases that cause a significant number of deaths worldwide. The time from exposure to the afore-mentioned adverse outcomes in regular animal-based testing is usually 14 to 28 or even up to 90 days post exposure. The disclosed methods can achieve this in less than few days, thus much ahead of time. Namely, it firstly uses cells, cell cultures and/or chip-like devices to deduce and parameterize the early evolution of biological systems, which is then in silico propagated to predict outcomes. The disclosed methods are thus based on evolution of molecular events that are the first to occur, long before they are shown in tissues as long-term effects.

In the methods of the present disclosure, these molecular events are analysed, measured and/or detected in a time-lapse manner (at least three time points) and in silico time propagation is performed, wherein material testing in only performed for up to 1 week, preferably up to 3 days, most preferably up to 50 hours, in order to correlate the early events to the long-term effects leading a reliable prediction about safety/toxicity of materials. Not all early molecular events are relevant for the adverse long-term effects; however, the in-silico time propagation is arranged to recognize only relevant early events.

An aspect of the disclosed methods is therefore an in silico forecasting of in vitro-detectable and in vivo relevant observables, which are propagated in time from their initial values [symbol] (t=0) using a system of differential equations. The in vitro detectable early observables' evolution can be automatically translated into set of parameterizable predefined interaction terms, which define the system of differential equations suitable for needed time propagation. These equations are preferably defined by the interaction terms of the interaction matrix [symbol] (t)=IM(t)· [symbol] , which is in turn determined during in vitro experiments using the concept of scenaria.

In one embodiment time derivatives of quantified observables are used to construct an interaction matrix that connects the vector of time derivatives of observables [symbol] ={\dot{q}ᵢ}=IM( [symbol] ) from a vector of observables [symbol] ={qᵢ} for a given scenario S={u₁, u₂, . . . }, where uᵢ are unity values 0 or 1 and represent whether individual observables qi are selected in the scenario S.

In another embodiment the interaction matrix term IMᵢⱼ comprises of a power parameter pᵢⱼ and at least two interaction function IFᵢⱼ that defines the dependence of an Interaction matrix term IMᵢⱼ on the observable qᵢ.

The method is not limited by the type of the chosen observables, their number and/or method how they were determined, calculated or detected.

Optionally, methods of the present disclosure comprise three main steps, namely:
a) Preparation phase to associatively identify the relevant observables, preferably on the basis of prior knowledge, AOPs, in vivo data as well as previous results obtained by the method,
In vitro experimentation, comprising the steps of:
b) Preparation of an in vitro model to prepare key event relevant and adverse associated living system, preferably chosen on the basis of expected function of living system, mode of action and/or point of entry, wherein the in vitro model comprises at least one cell type,
c) Scenaria-based time-lapse acquisition with to detect early time evolution of the observables in the in vitro model, with minimally 3 time points at an arbitrary time delays longer than 5 min (corresponding to the fastest biological context change) and total duration of up to 1 week (preferably less than 2 days; corresponding to the longest interval that enables reliable determination of events' evolution), within minimally two scenaria, wherein a scenario is a subgroup of measurable observables that are structural, chemical, biological, mechanical, physiological, immunological and similar events, wherein at least one scenaria refers to unexposed in vitro model and at least one for exposed in vitro model, with tested substance applied in a dose that is able to induce at least minimal effect within the in vitro model (in toxicology referred as LOAEL-least observable adverse effect level),
d) Quantification of the observables to quantify their values within in vitro model, wherein at least one observable refers to a property of an unexposed in vitro model and at least one observable relates to the property related to the exposure-related changes within an in vitro model,
e) Generation of time series and time derivatives of the observables to obtain a vector of observables for the scenaria, which define the time evolution of the in vitro model and the comprising events,
Mechanism determination, comprising the steps of:
f) Construction of the interaction terms of interaction matrix through the interaction functions to biophysically and biologically define all possible interactions and couplings between the observables, which in turn mathematically determinate the time evolution of all of the observables,
g) Construction of base functions and super-equations to translate set of interaction functions from previous step into mathematically orthogonal set of functions, that can be used to numerically parameterize the evolution of observables in the next step,
h) Parameterization of the interaction terms that define the rates, by which observables change, the parametrization using scenaria-derived experimental data, previously defined orthogonal set of functions and selected numerical methods to determine the system of equations and enable numerical time propagation of the observables in the next step and identification of the most relevant interaction terms in the second next step,
i) Scenario-dependent fit to in vitro experiments to use parameterization of the interaction matrix from the previous step and derive values of each of the observables for each scenario starting from the given initial condition for each observable,
j) Optional presentation of the determined mechanism to identify the most relevant interaction terms (mode-of-action) and illustrate their contribution to evolution of the observed early events-observables for a tested agent and their development into a potential adverse outcome,
Outcome prediction based on the results obtained with the above-mentioned steps, wherein the prediction may optionally further include the steps of:
k) Optional dose biodistribution characterization, to replace costly animal-based determination of dose-related information such as no-observable-adverse-effect-level (NOAEL dose) and least-observable-adverse-effect-level (LOAEL dose), preferably by in vitro-based image-derived bio-distribution,
l) Dose-dependent long-time propagation of observables to finally employ parameterized interaction matrix (determined mode of action) in propagating the AO-related observables for a long time to determine AO-prediction much before AO could evolve and would be observed in animal testing.

In the method described by Kokot et al., observables (events) were not grouped, description of a model, responsible for propagation of events in time, relied on predefined differential equations and interaction terms-two single in vitro and one in vivo measurement were used in prediction that is based on a pre-known mechanism which was pre-discovered for a known material. The mechanism then allowed creation of a nomogram-based parametrization, which already considered in silico time propagation. It was impossible to apply the prediction to materials for which mathematical model has not evolved from mode-of-action.

On the contrary, the presently disclosed methods represent an upgrade of the method by Kokot et al. by excluding the need for knowledge and understanding of the mechanisms of material toxicity, cellular response and the connection between individual early cellular events. While the prediction enabled by Kokot et al. method can only be used for the materials that evolve toxicity by exactly the same pre-discovered mechanism with all the interaction terms for all the observables related to pre-defined in vitro model, the current methods utilize knowledge about which observable is in vivo relevant and in vitro observable. In the present methods, the time propagation of the key events (in vitro system), used in prediction of outcome, is achieved with a general system of differential equations, which are parameterized later automatically using the concept of the interaction matrix, the set of scenarios, associated to individual experiments and used to include/exclude individual observables, as well as the specially derived algorithm that is able to translate time derivatives of observables into power parameters of individual interaction matrix elements (interaction terms).

These and other objects, advantages, purposes and features of the disclosed methods will become apparent upon review of the following specification in conjunction with the drawings.

### BRIEF DESCRIPTIONS OF THE DRAWINGS

Methods for predictive testing of agents will be disclosed in further detail based on non-limiting exemplary embodiments with reference to the accompanying drawings, in which:

FIG. 1A schematic view of the alternative testing methodology in comparison to the animal-based endpoint-oriented testing;

FIG. 2 An overview of the key steps of the method a)-l);

FIG. 3A non-exclusive list of possible in vitro models used in Step b);

FIG. 4A non-exclusive list of possible methods for time-dependent data acquisition in Step c);

FIG. 5 Example of image quantification process with 2 observables related to cell properties and 3 to agent (toxicant) surface used in Step d);

FIG. 6 Possible interaction functions and their parameterization used in Step f) with all biologically relevant and numerically applicable interaction functions grouped within the dashed region and the two additional interaction functions used in the approximation of the interaction function of the quotients grouped within the dotted region;

FIG. 7A generalized interaction matrix with 9 blocks indicated together with typical interaction term expressions and interaction functions used in Step f);

FIG. 8 Exemplary fit to the early 30h evolution of events that correspond to the exposure with two selected materials to the lung alveolar epithelium with all of the observables indicated with callouts as appeared in Step i);

FIG. 9 Graphical pictograms used in mechanism presentation of Step j);

FIG. 10 Parameterization-based mechanistic presentation of TiO₂ nanotube action used in Step j), with triple encoding of the significance—the insignificant effects appear almost transparent, grey and thin, change in color form green to magenta represents the change from positive to negative effect, quantities that are affected lie in the inner circle, while the quantities that cause the effect lie on the outer circle, self-stimulating and self-inhibitory effects are depicted with semi-circular arrows;

FIG. 11 Parameterization-based mechanistic presentation of multi-wall carbon nanotubes action used in Step j);

FIG. 12 Calculation of image-based dose biodistribution as used in Step k) illustrated on the case of two channel image analysis that serves for determination of toxicant-surface-to-membrane-surface dose;

FIG. 13 Expanded interaction matrix with additional part not defined through in vitro system but via AOP knowledge and other available in vivo-related data indicated with dotted region as used in Step l);

FIG. 14A representative example of the actually acquired time-lapsed 3-channel (2 fluorescence and 1 back-scattered) images of TiO₂-exposed in vitro lung model as a part of data acquisition in Step c);

FIG. 15 An example of the interaction matrix with the interaction functions implemented for the case of chronic inflammation prediction of TiO₂ nanotubes-exposed in vitro lung model as a part of Step f);

FIG. 16 Simplification of the interaction matrix in Step f) with nulled interaction terms indicated by zeroes, excluding e.g. biodegradation/biosynthesis (in block D), adsorption/desorption from phenomena-related structures (in block B), interference in growth between the cell lines (cross-terms in the left upper block of block G), indirect toxicity (cross terms in the central upper block of block T), and relocation/direct exchange of the toxicant between the cell lines (in the central block block R);

FIG. 17 An example of the simplified interaction matrix with the interaction functions implemented for the case of chronic inflammation prediction of TiO₂ nanotubes-exposed in vitro lung model as a part of Step f);

FIG. 18 An example of the base functions and associated parameters' list corresponding to the simplified version of the interaction matrix for the case of chronic inflammation prediction of TiO₂ nanotubes-exposed in vitro lung model for each of the 6 scenaria as appeared in Step g);

FIG. 19 Parameterization of the generalized interaction matrix (A) and simplified interaction matrix (B) used in prediction of TiO₂ nanotubes and derived by the SVD showing the effect of additional constrains in boxes (positive definiteness of parameters in Block R);

FIG. 20 An example of the final result (of Step l) including dose distribution histogram, 800 h long-time propagation of all observables indicated with callouts and long-time prediction of chronic-inflammation-related observable (macrophages) relevant for in vitro lung model exposed TiO₂ nanotubes, where left and right graph (bottom) correspond to the smallest and the largest local doses from the dose histogram (top), respectively; and

FIG. 21 An example of the 2- and 3-channel microscopic images used to predict adverse outcomes for 3 various agents: vaccine based on virus-like particles, micro- and nanoplastics, drug and food supplement with cells compartment always shown in green channel and agent (possible toxicant) channel indicated with colour of the text on the image.

### DETAILED DESCRIPTION

Methods of the present disclosure provide means to shift from the current toxicity testing concept, which relies on adverse outcome testing in animals, to the new testing, which can rely on observation of early events that follow immediately after the molecular initiating event (first response to an exposure) and occur much earlier than adverse outcome. As such, the disclosed methods can significantly shorten the time needed for the test to be done.

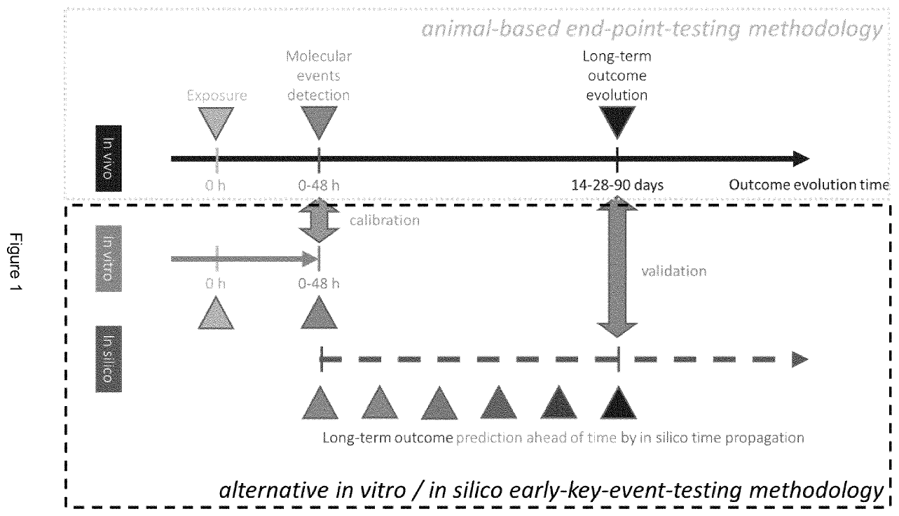

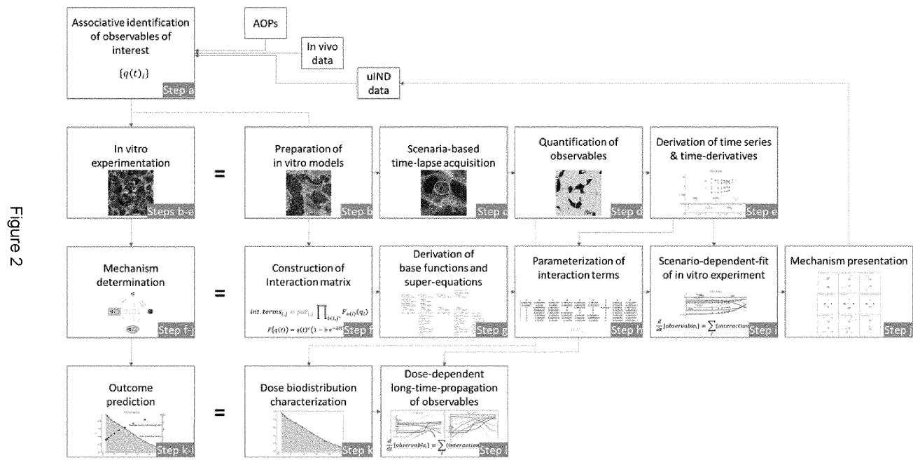

FIG. 1 shows a comparison of the time required for testing between known methods of (animal-based) in vivo testing, wherein long-term outcomes are analyzed only after 14, 28 or 90 days upon exposure, while the disclosed method allows detection of early molecular events indicative for the long-term outcomes much earlier, for example up to 48 hours upon exposure.

One method for predictive testing comprises of twelve well-defined steps, some of which are optional and are included to improve the reporting to the users of the testing. Implementation of entire methods is exemplary in a non-limiting manner demonstrated in Examples 1 and 2. Variety of implementations in individual steps are demonstrated in Examples 3, 4 and 5.

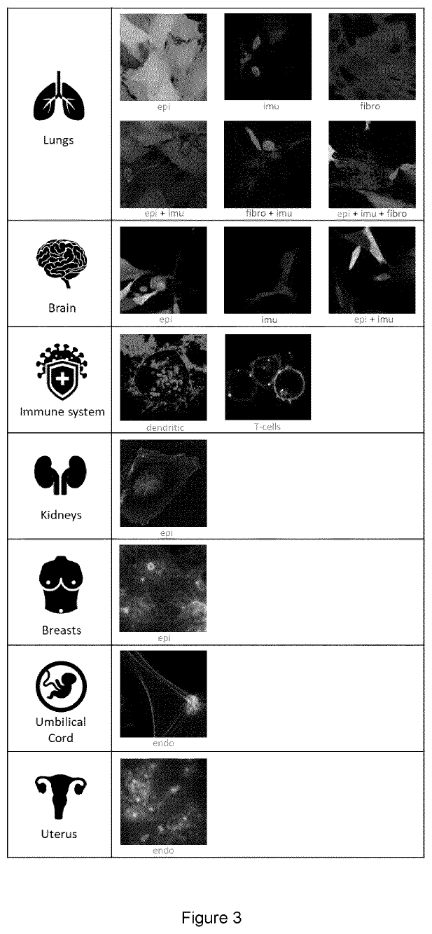

FIG. 2 shows an embodiment of the method for predictive testing, wherein the following steps are performed:
a) associative identification of relevant observables on the basis of prior knowledge, AOPs, in vivo data as well as previous results obtained by the method,
In vitro experimentation, comprising the steps of:
b) Preparation of a suitable in vitro model based on in step a) identified observables and knowledge,
c) Scenaria-based time-lapse acquisition of events in step b) prepared in vitro model, with at least three time points at an arbitrary time delays longer than 5 min, within minimally two scenaria, wherein a scenario is a group of measurable observables that are structural, chemical, biological, mechanical, physiological, immunological and similar events,
d) Quantification of the observables to quantify their values within in vitro model, wherein at least one observable refers to a property of an unexposed in vitro model and at least one observable relates to the property related to the exposure-related changes within an in vitro model, for all of which observables are acquired in step c),
e) Generation of time series and time derivatives of the observables quantify in step d) to obtain a time evolution of observables for the scenaria,
Mechanism determination, comprising the steps of:
f) Construction of the interaction terms of interaction matrix through the interaction functions to biophysically and biologically define all possible interactions and couplings between the observables, which in turn mathematically determinate the time evolution of all of the observables by providing the most generalized description of relations between the time derivatives of observables obtained in step e),
g) Construction of orthogonal base functions and super-equations for interaction matrix from step f) using all scenaria-associated combination of variables monitored in step c),
h) Parameterization of interaction terms using definitions from step g) and time-dependent values and derivatives of observables from step e), obtained parameters define the rates, by which observables change, the parametrization using scenaria-derived experimental data, previously defined orthogonal set of functions and selected numerical methods to determine the system of equations and enable numerical time propagation of the observables in the next step and identification of the most relevant interaction terms in the second next step,
i) Scenario-dependent fit to in vitro experiments using initial values of observables from step d) and e) and time evolutions from step f) with parameterization determined in step h) for each scenaria used in step c),
j) Optionally mechanism presentation using parameterization derived in step h), and
Outcome prediction based on the results obtained with the above-mentioned steps, wherein the prediction may optionally further include the steps of:
k) Dose biodistribution characterization using image-based dose biodistribution analysis from images acquired in step c),
l) Dose-dependent long-time propagation of observables identified in step a), interaction matrix parameterized in step h) and observables evolution derived in step j) for each of the doses of the distribution histogram determined in step k).

The final user-friendly and understandable results of the method for predictive testing of agents according to the disclosed methods may be delivered by Step j), Step k) and Step l).

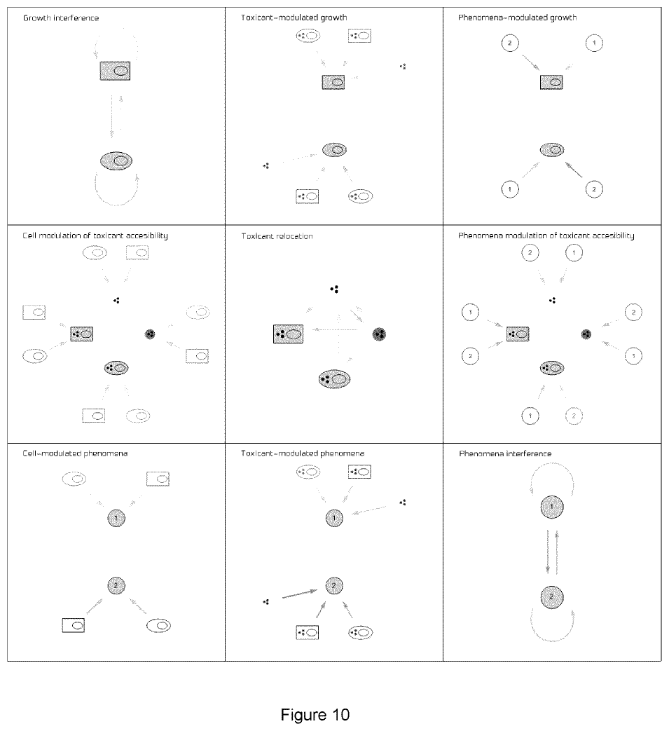

The results of Step j) that describe the mechanism (mode of action) may be represented by graphical material similar to parts of FIG. 10 and may comprise of the following statements:
Agent T is severely toxic to cell type Ci
Agent T is not toxic to cell type Ci
Agent T exhibits contact toxicity to cell type Ci
Agent T is indirectly toxic to cell type Ci if uptaken in cell type Cj
Local phenomenon Pii stimulates/inhibit growth of cell type Cj
Cell type Ci increases/decreases accessibility of agent T in compartment Cj
Cell type Ci induces aggregation/biodegrades of agent T in compartment Cj
Agent T is efficiently uptaken by cell type Ci
Agent T is efficiently uptaken by cell type Cj from cell type Ci
Agent T is being quarantined
Phenomenon Plj increases/decreases accessibility of agent T in compartment Cj
Phenomenon Plj induces aggregation/biodegrades of agent T in compartment Cj
Agent T stimulates/inhibits phenomenon Pli when being in cell type Cj
Agent T stimulates/inhibits phenomenon Pli when being quarantined
Phenomenon Pli stimulates/inhibits phenomenon Plj
Phenomenon Pli self-stimulates/self-inhibits its action

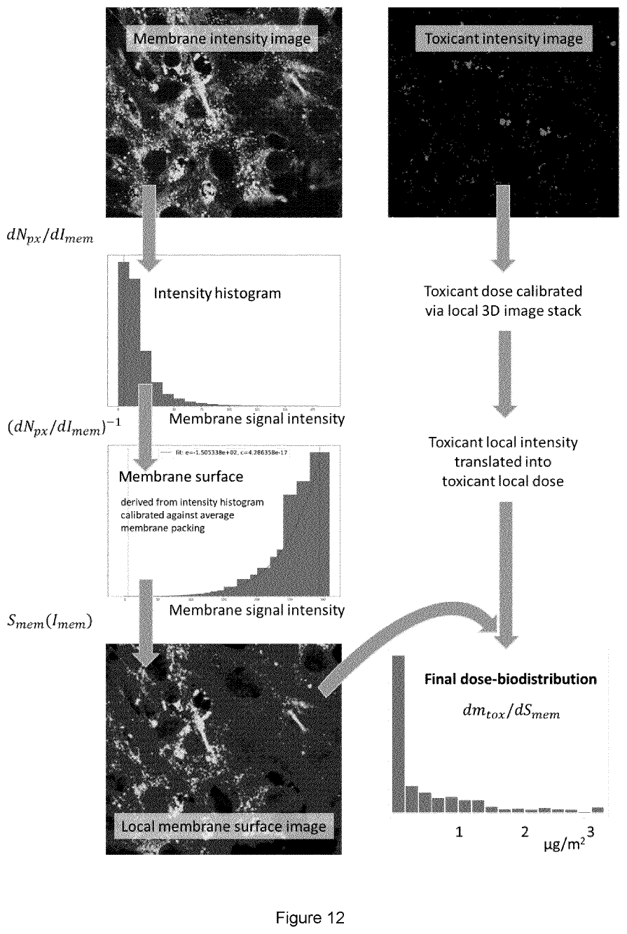

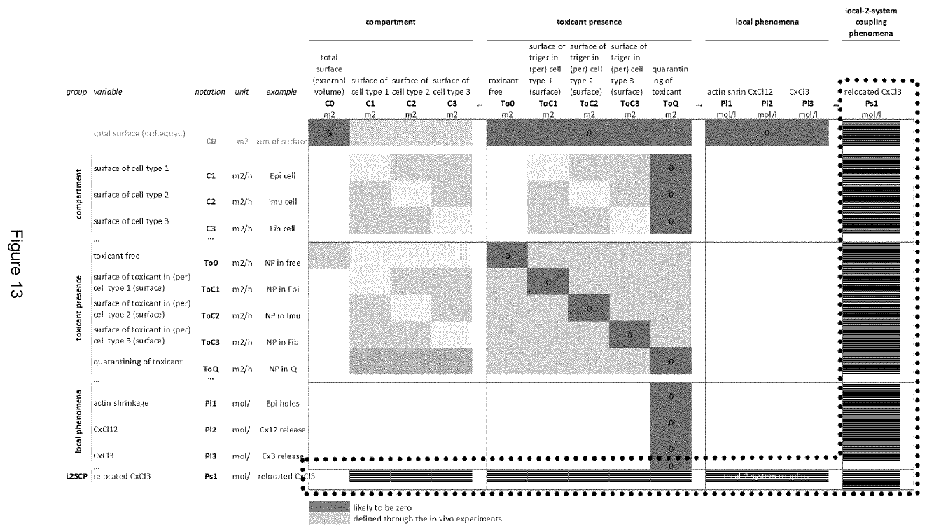

The results of Step k) that describe the biodistribution of agent, may be represented by graphical material similar to parts of FIG. 12 and may comprise some of the statements form Step j) and/or the following statements:
Agent T is homogeneously dispersed within in vitro model in different sizes, indicating that the biological system favors dispersed forms of agent T and unfavors its aggregated states
Agent T is detected in large aggregates, indicated that the biological system favors aggregated states of agent T
Agents T is efficiently quarantined on/in/beside/outside of cell type Ci
Agents T is not uptaken by the cell type Ci
Agents T is dispersed in forms of many small sub-micron-sized particles in cell type Ci

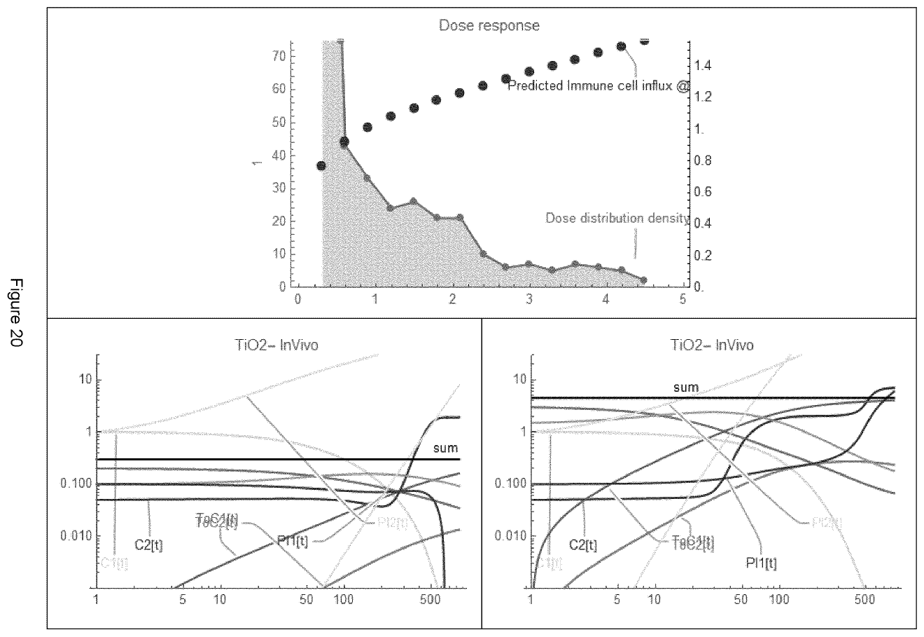

The results of Step l) delivers final adverse outcome prediction associated with the agent, may be represented by graphical material similar to parts of FIG. 20 and may combine the statements from Steps j) and k), add additional statements to further define evolution of biological systems, and/or provide other kind of statements. It may comprise of the following statements:
Agent T is safe-biological system does not respond with any abnormal evolution
Agent T is safe-agent T does not trigger any delayed response
Agent T is safe at the doses, which may be expected to be encountered at the specific conditions
Agent T is safe at doses smaller than specific values
Agent T is safe at dose rate smaller than specific value
Agent T triggers weak/strong/extra strong acute response (above certain dose or dose rate), which however resolve in specific time
Agents T triggers an acute response (above certain dose or dose rate), which is (above certain different dose or dose rate) later on amplified into strong subacute/sub-chronic response with the potential to transform into long-lasted chronic inflammation
Agent T instantly causes severe cell damage, reflecting its huge toxicity
Agent T triggers weak but long-lasting response in terms of triggering constant elevated phenomena/immune response
Agent T triggers weak acute response, which later on however amplifies into non-resolvable constantly-amplifying inflammation (cytokine storm)

The steps of the method for predictive testing are implemented as follows:

### a) Associative Identification of Relevant Observables

The choice of observables depends on the tested substance and pre-existing knowledge on development of possible substance-related outcomes (symptoms).

For example, at least a part of observables can be selected based on AOPs, previous in vivo data and/or scientific reports and articles. At least one of the observables should preferably match the earliest event that is observed in vivo or in patients, if adverse outcome evolution is studied.

For example, in case when toxicity assessment deals with inhalation and lungs are the organ of interest, because it is exposed first, observables related to chronic inflammation are related with immune cells, for example:
the number of monocytes that enter the lungs (because they differentiate into macrophages replacing dead residential macrophages),
the number of polymorphonuclear (PMN) cells that enter the lungs (because it is most convenient to identify them ex vivo, and they correlate well with influx of the monocytes),
the number of leukocytes that enter the lungs (because their time dependence rate changes most significantly and correlates well with PMN influx as well).

### b) Preparation of a Suitable In Vitro Model

The choice of the in vitro model thus first depends on the observables that are required to reproduce in vivo relevant events and which are selected in Step a). The later might depend on tested substance as well, for example inhaled particles are tested on in vitro models relevant for lungs (either bronchial or alveolar part, depending on size of substance), comprising epithelial lung cells and/or lung macrophages and/or fibroblasts, and/or endothelial cells and/or other immune or other type(s) of cells.

The in vitro model may comprise any combination of single cultures of cells or various co-cultures of cells or more complex cell cultures depending on the specifics of tested substances. Cells for the in vitro model may thus be selected from a group consisting of at least epithelial lung cells, epithelial skin cells, neurons, endothelial cells, muscle cells, intestinal epithelium cells, mucous cells, parietal cells, chief cells, endocrine cells, immune cells (macrophages, glia, . . . ), etc.

The skilled person is aware of the suitable relevant combination of cells for analysis of particular substances. Growth media and growth conditions are optimized with regards to the chosen in vitro model mimicking a relevant tissue and supporting growth of the selected combination of cells.

Identification of observables (in Step a) therefore directly affects the selection of cell types that constitute the chosen in vitro model. The construction of an in vitro model therefore comprises the following requirements:
The chosen in vitro model is relevant to the particular agent assessment, mimicking at least a relevant part of the relevant target tissue(s) as well as the relevant agent delivery path,
every cell type or their combination used
   structurally or functionally mimics AOP-relevant part of the targeted tissue, and
   is able to express one or more AOP-relevant key events identified from prior knowledge;
all cell types together are able to respond with a relevant early part of AOP
   preceding the later part of the AOP, which can be observed in vivo and which develops into observed adverse effect, and
   leading to at least one key event that has the power to predict the adverse outcome,
      wherein AOP refers to generalized concept of Adverse Outcome Pathway that comprises all the causally connected events from the Molecular initiating event and all the following events to the final adverse outcome, independently whether the event is physical, chemical, biological, biophysical, biochemical or of any other type.

In Example 3 and in FIG. 3 a non-exclusive list of possible in vitro models, that are relevant for various types of in vivo events (chronic inflammation, neurodegeneration, cancer, and other slowly evolving diseases of various organs) to perform Step b), are shown.

### c) Scenaria-Based Time-Lapse Acquisition

After observables are identified (Step a) and proper in vitro model is constructed to enable mimicking the molecular initiating event and its further early evolution (Step b), methods for detecting such molecular events needs to be identified and selected.

Methods to acquire observables of interest within the living in vitro model may be any suitable and widely used methods, preferably in the time-lapse mode with required time resolution to capture the dynamics, typically on the order of a few minutes to hours, such as:
Microscopy, such as brightfield, fluorescence widefield or confocal, scattering, Raman, ESEM, FTIR, etc. to enable observation and quantification of time-evolution of the observables (related to structures or biochemical information, see below) and determination of their time derivatives;
spectroscopy, such as Raman, FTIR, UV-VIS, fluorescence, specific staining and similar to enable determining the concentrations/amounts of the compound classes of interest in entire samples (e.g. of proteins, lipids, aromatic substances, labelled substances, tagged substances, inorganic substances) and quantification of their time-evolution and determination of their time derivatives;
Immunological methods, such as ELISA, antibody isolation and purification, ELISPOT, immunoblotting, immunohistochemistry, immunoprecipitation, immune cell isolation, etc., to enable determining the concentrations/amounts of the specific compounds of interest in entire samples (e.g. of the selected cytokines) and quantification of their time-evolution and determination of their time derivatives;
Omics data such as proteomics, lipidomics, transcriptomics, metabolomics, etc.

Observed early events (observables) can be any effects that the tested substance has on cellular system, for example change of molecular (for example lipid, protein, RNA, DNA, etc.) or supramolecular (for example membrane, ribosome, cytoskeleton, fibres, vesicles, etc.) or cellular (cell surface, volume, shape, activity, etc.) property that can be quantified, wherein the events are usually selected from the group consisting of:
changes in morphology, shape, and mobility of cells,
aggregation and/or dissociation of cells,
altered gene expression,
increased or decreased lipid expression,
increased or decreased transport of molecules and other structures into or from the extracellular space (vesicles, etc),
disconnecting contacts between cells (tight connections),
actin and tubulin rearrangement,
chromatin (de) condensation and other nuclear phase changes,
cell lysis,
apoptosis or necrosis events or events related to other cell state changes,
changes in ER, endosomes, lysosomes, mitochondria and/or ribosomes,
cell death,
quarantining, phagocytotic or other new structure formation on the surface of cells or inside cells, for example quarantine being described by Kokot et al.,
transcription, synthesis, expression, relocation and release of attractants from immune and other type of cells,
binding state, charge, interaction surface chemical composition of molecules, molecular complexes and supramolecular structures, as well as aggregated structure (endogenic or exogenic),
type of interaction exhibited by the interaction surface by any cellular organelle or molecular complex, supramolecular structure or external/exogenic materials,
release and/or absorbance of a molecule, vesicle, etc from one inter/intra-cellular compartment into another,
Surface area of cells of the chosen type (corresponds also to number of cells or concentration of cells, etc.),
Surface area of the agent (toxicant) in a chosen compartment (corresponds also to mass, concentration, volume, etc.)
Amount of signalling or transporting molecules or other structures (corresponds also to mass, concentration, binding state, etc. of cytokines, chemokines, enzymes, receptors, RNA of various types, exosomes, endosomes, lipid bodies, etc.),
Surface area of new or modified structures (corresponds also to mass, volume, density, lifetime, dimensionality, correlation lengths, shape descriptors, etc. of supramolecular structures such as vesicular structures, plasma or internal membranes, fibres inside or outside the cells, tight junctions between the cells, gaps between the cells, mitochondria or mitochondrial network, nucleus or parts/domains in nucleus, etc.).

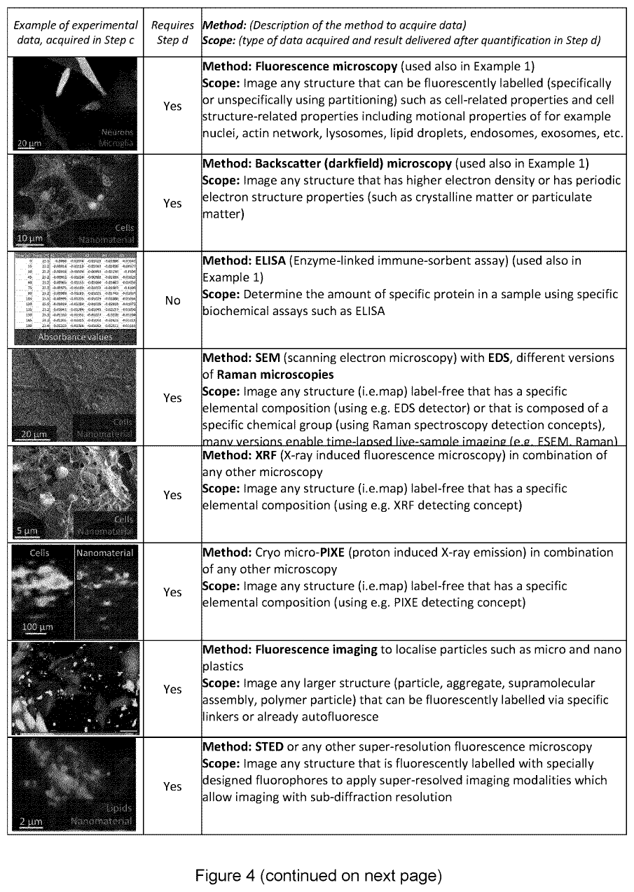

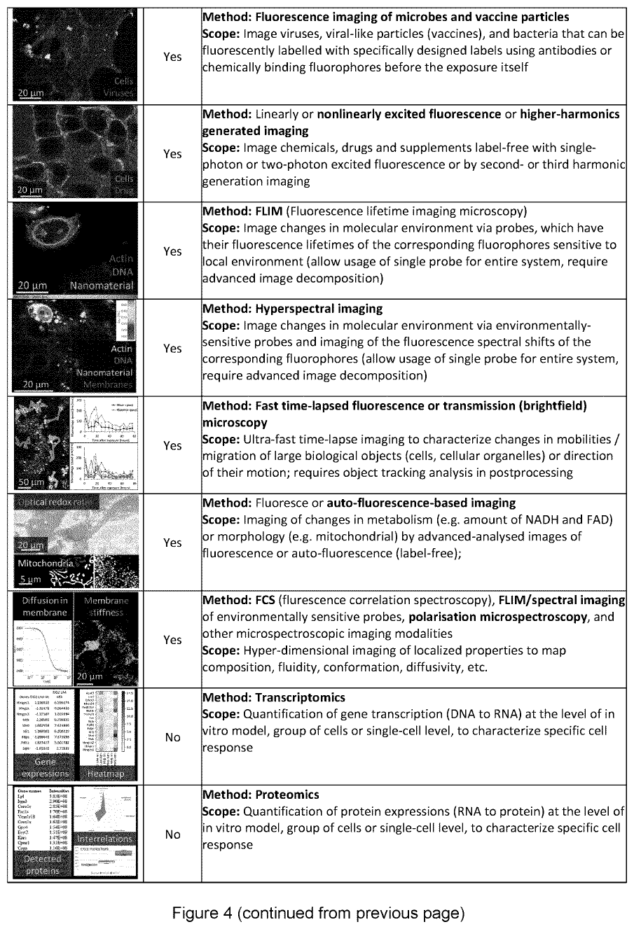

In Example 4 and in FIG. 4 a non-exclusive list of possible methods, that enable time-dependent data acquisition to perform Step c), are shown.

### d) Quantification of the Observables

Quantification of observables in control (no substance) and exposed (tested substance applied in a selected dose) in vitro models depends on the type of the acquired data. If the latter are already in a numerical form (e.g. ELISA, transcriptomics, proteomics), it is directly passed onto the next step of derivative generation, otherwise, for example when images were acquired, the analysis performs a two-step method of:
masking the parts of images that associate with the events (observables), for example type of cell lines, labelling of cells or cellular structures when appropriate, sensitivity of the methods, colocalization information, etc, and
quantification of the corresponding observable, wherein the masks from the previous step are translated into quantified observable q at each time t and each region of interest (ROI), wherein normally, the surface areas are obtained by summing up the number of pixels within the mask and the amounts are derived by summing up the intensities of the appropriate image channel(s) within the mask.

Optionally, additional histogram or intensity-distribution analysis (like FIDA, fluorescence-intensity distribution analysis) is used to further sharpen the quantification procedure. Quantification results from multiple ROIs can be summed up (like surface area of cell types or particular structural response), effectively increasing the field-of-view of a single experiment and thereby decreasing the noise (stemming from local variability) in the analysis.

For illustrative purposes, the surface area of a selected cell type or a selected structural type is determined as a number of pixels assigned to the selected cell type or selected structural type, for which a binary mask of the corresponding cell/structure is needed. In an image with one cell type and a single expressed event, such a mask is trivially derived by binarizing the image. In an image of a more complex cell cultures, such as coculture, or cultures that can express several structural or biochemical events at the same time, multi-labelling is often used (colocalization or off-localization) together with morphological information (sizes, dimensions, shapes, etc.) and information from the z-stack (localization above and below the analyzed image) to discriminate different events and obtain several masks from the same image. Optionally, when the quantity is associated with a structure exhibiting certain geometrical or morphological properties, such as size, aspect-ratio, density, bulk or surface of a structure, etc., the mask is segmented by one of the available image segmentation algorithms and each segmented object is analyzed in terms of the desired property. Accordingly, the mask is reduced to incorporate only those (parts of) objects that meet the desired criteria. For example, the mask of the quarantined agent-toxicant (e.g., material that becomes inaccessible for cellular sensing mechanisms or cellular processing) is derived by binarizing the intensity of the channel corresponding to the material, followed by segmentation to find aggregates of sizes above a threshold and final removal of the pixels of the mask that correspond to the surface of the segmented objects.

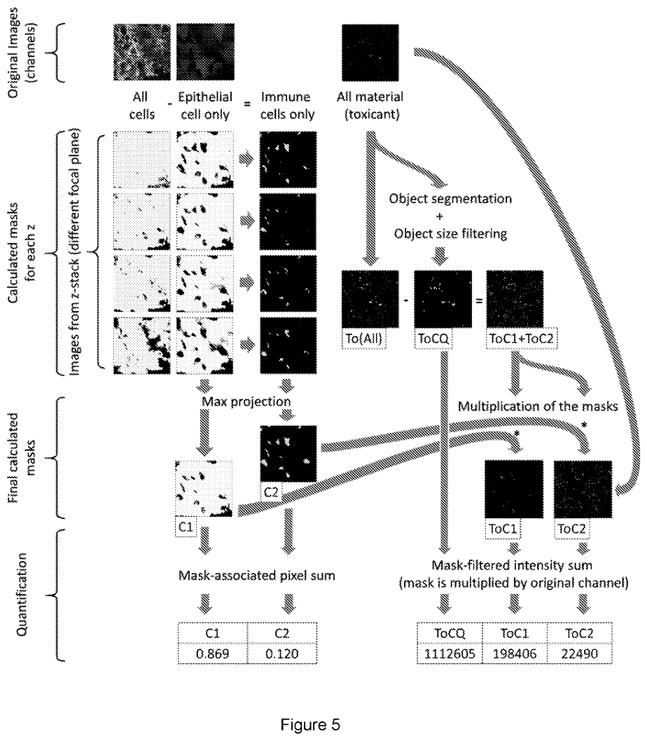

In Example 5 and in FIG. 5, 5 possible quantification processes are presented to nonexclusively illustrate how the images are processed and observables quantified.

### e) Generation of Time Series and Time Derivatives

Once observables are quantified in all measured and analyzed time points, derivation of the time series and time derivatives of observables is performed in order to deliver time-dependent observables and time-dependent time derivatives of observables. The latter is preferably done in a noise-decreasing way taking the information from all the available time points and ROIs to improve certainty of results due to limited field-of-view, cell migration during time-lapse monitoring, changes in labelling and labelling efficiency during monitoring, limited number of observed key events, detection noise, uncertainty generated during masking, segmentation, etc.

Noise-decreasing way of time derivative determination involves the following assumptions:
each of the observables (key events) changes mostly slowly in time, with at most one maximum or minimum in the time interval of experimental monitoring,
each of the observables can have at most one segment in which it changes fast in the time interval of experimental monitoring, meaning that the absolute value of the observable's derivative has at most one maximum or minimum,
any other fast changes are due to experimental noise.

With that in mind, the set (i.e. vector, denoted by { }) of measured values {qᵢ(tⱼ)} of observables qᵢ at times tⱼ, within the given time interval of experimental monitoring {t₁ . . . tₘₐₓ} can be approximated with a set of fitted values {q_{i-f}(tⱼ)} (additional index-f indicates fitted values) or interpolated by fitting {qᵢ(tⱼ)} to a predefined function such as:

$${q}_{i - f} ( t ) = a + bt Exp [ ct ] \qquad [ 1 ]$$

Analogously, time set of observable's derivative values {dqᵢ/dt(tⱼ)} is approximated with a set of derivative values {dq_{i-f}/dt(tⱼ)} at the same time points {tⱼ}.

When referring to a set of experimental data {{tⱼ}, {qᵢ(tⱼ)}, {dqᵢ/dt(tⱼ)}} in the following steps of mechanism determination, especially formation of the interaction matrix and parametrization, the following values of time points, observables and their time derivates at given time points are used as an input

$$\{ \{ {t}_{j} \} , \{ {q}_{i - f} ( {t}_{j} ) \} , \{ \frac{{dq}_{i - f}}{dt} ( {t}_{j} ) \} \} . \qquad [ 2 ]$$

### f) Construction of the Interaction Matrix

Construction of interaction matrix, wherein (long-) time-propagation of observables qᵢ is achieved by using a system of differential equations, schematically depicted as follows

$$\frac{d}{dt} [ {observable}_{i} ] = {\sum }_{j} {( interaction terms )}_{ij} \to \frac{d}{dt} {q}_{i} = {\sum }_{j} {IM}_{ij} \to \dot{\vec{q}} = \{ {\dot{q}}_{i} \} = IM ( \vec{q} ) \qquad [ 3 ]$$

where the interaction terms define the rates that cause the change of the observable qᵢ, resembling the interactions between the chosen observable qᵢ and other observables qⱼ. In essence, each interaction matrix element (interaction terms IM; in Eq[3]) is defined as a product of a power parameter and the product of interaction functions of observables related to qᵢ and qⱼ and defined with (i,j)-specific derivatives o

$${IM}_{i , j} = {p}_{i , j} {\prod }_{k ∈ i , {j}^{*}} {IF}_{o ( k )} ( {q}_{k} ) \qquad [ 4 ]$$

with power parameter p_{i,j} defining the influence of each interaction term to the change rate of the observable qᵢ. The product, as it will be seen later, can generally involve more than 2 interaction functions, which are however always related to the indexes of observables i and j.

In some cases, where the interaction function depends on the quotient of two observables q and r, for example in concentrations (the amount or surface area of the agent (toxicant) per volume of the compartment), it is desirable to transform such an interaction function of a quotient into a product of two interaction functions where one depends on first observables and the other depends on the second observable:

$$IF ( \frac{q}{r} ) = IF ( q ) IF ( r ) + O ( q , r ) ≅ IF ( q ) IF ( r ) \qquad [ 5 ]$$

As it will be shown below, in practical cases the remnant O(q,r) represents less than 20% of the original value, and the above approximation will be considered satisfactory.

To generalize the interaction function IF(q) to take a form independent of the (pre)known mechanisms, the following dependence of the interaction term on the observable q is assumed

$${IF}_{o} ( q ) = IF ( q ( t ) , a , b , c ) = {q ( t )}^{a} ( 1 - {be}^{- {q ( t )}^{c}} ) = IF ( q , o 1 , o 2 ) \qquad [ 6 ]$$

where a, b and c can have simple integer values 0, 1, 2, . . . and can be translated into o₁ and o₂, which have integer values of −1, 0, 1, 2 . . . and define the derivates dIF/dq at small and large values of q, i.e. determine the sensitivity of the interaction term to smaller and/or higher values of the observable q. Because the sensitivity is now parameterized, it can be either fitted from in vitro data or predefined based on general knowledge.

The set (a,b,c) and (o₁, o₂) from Eq. [6] are related via the following relations:

$$\begin{matrix}\begin{matrix}Σ = o 1 + o 2 ; \\ a = o 2 ; \\ b = 2 - Σ - 1 / 2 ( \vert 1 - Σ \vert + 1 - Σ ) ; \\ c = 2 - Σ ;\end{matrix} & \begin{matrix}o 1 & o 2 & a & b & c \\ 0 & 0 & 0 & 1 & 2 \\ 0 & 1 & 1 & 1 & 1 \\ 0 & 2 & 2 & 0 & 0 \\ 1 & 0 & 0 & 1 & 1 \\ 1 & 1 & 1 & 0 & 0\end{matrix}\end{matrix} \qquad [ 7 ]$$

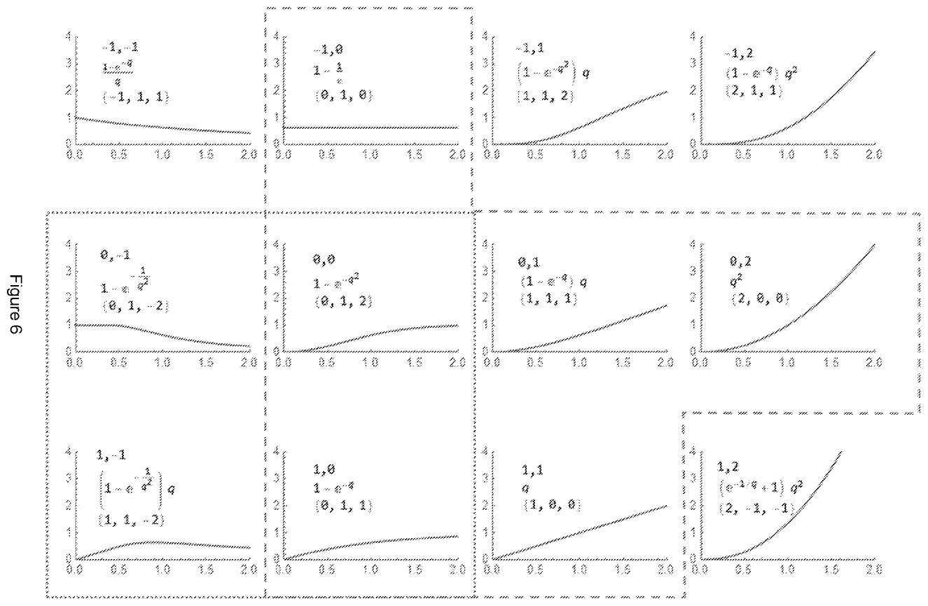

The following 6 interaction functions (indicated with the dashed region in FIG. 6, and identified below by the pair (o₁, o₂) represent the most biologically relevant interaction functions (influencing its rate):
(1,1)—linear dependence on q,
(1,0)—saturated dependence on q,
(0,1)—thresholded/delayed dependence on q,
(0,0)—saturated & thresholded dependence on q,
(0,2)—nonlinear/quadratic dependence on q,
(−1,0)—term independent of q,

The additional two interaction functions (indicated within the part of the dotted region in FIG. 6 that extends beyond the dashed region) represent the two functions that are used in the approximation of the interaction functions with the quotient of two observables q and r mentioned in Eq. [5]:

$$( 1 - Exp [ - {q}^{2} / {r}^{2} ] ) ≅ ( 1 - Exp [ - 2 / {r}^{2} ] ) ( 1 - Exp [ - 2 {q}^{2} ] ) \qquad [ 8 ]$$

$$r ( 1 - Exp [ - {q}^{2} / {r}^{2} ] ) ≅ r ( 1 - Exp [ - 2 / {r}^{2} ] ) ( 1 - Exp [ - 2 {q}^{2} ] ) \qquad [ 9 ]$$

By having functionalized the interaction functions through Eq. [6] and parameterized their sensitivity through Eq. [4], the interaction terms from Eq. [4] need no longer to be defined in advance (as in Kokot et al.), but can in principle be determined by inverse problem-solving procedure using time-dependent experimental data.

The previously-mentioned scenarios are used as a tool to include/exclude observables that are relevant/irrelevant in each of the experiments. This reduces the list of interaction terms in each of the equations appropriately for each scenario. Mathematically, this is done by rewriting the set of differential equations in a matrix form

$$\frac{d}{dt} [ \begin{matrix}surfaces \\ toxicants \\ phenomena\end{matrix} ] = [ \begin{matrix}⋱ & ⋮ & ⋰ \\ … & interaction terms & … \\ ⋰ & ⋮ & ⋱\end{matrix} ] [ \begin{matrix}⋮ \\ scenaria \\ ⋮\end{matrix} ] \to \dot{\vec{q}} = \{ {\dot{q}}_{i} \} = \underline{IM ( t )} \cdot \vec{S} \qquad [ 10 ]$$

where scenaria are defined as vectors of zeroes and ones with the dimension of number of observables. Multiplication of the interaction matrix with a scenario vector eliminates those interaction terms in the differential equations that are not present in the particular experiment.

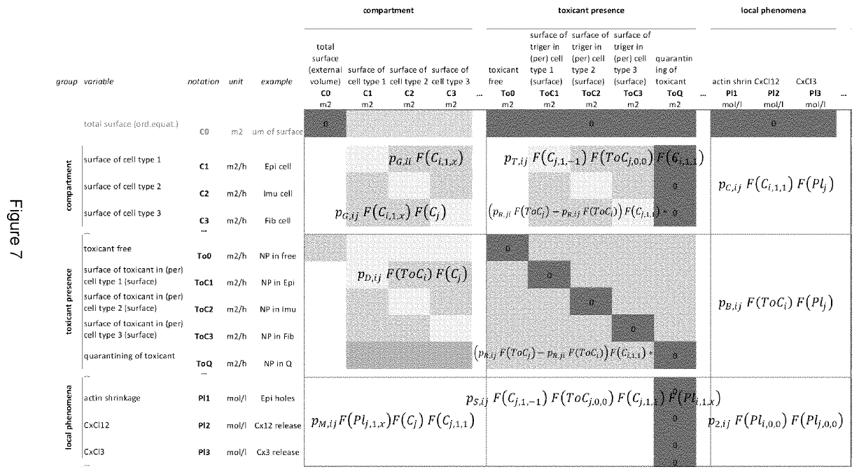

For exemplary purposes, demonstrated also in FIG. 7, observables q are sub-grouped into 3 classes: cell surface areas (compartments), amounts of agents, i.e. toxicants (associated with compartments), and occurrence frequencies of chosen phenomena, that logically lead to evolution of the interaction matrix-sub-grouping of the interaction matrix terms into 3×3 block form, where each block of the interaction matrix is associated with the following content (see blocks indicated in FIG. 7):

| | | |
|---|---|---|
| (G) - Growth | (T) - Toxicant- | (C) - Phenomena- |
| interference | modulated growth | modulated growth |
| (D) - Cell modulation | (R) - Toxicant | (B) - Phenomena |
| of toxicant | relocation | modulation of |
| accessibility | | toxicant accessibility |
| (M) - Cell-modulated | (S) - Toxicant- | (2) - Phenomena |
| phenomena | modulated phenomena | interference |

Beside the grouping of the interaction terms, the FIG. 7 indicates also the most probable classes of the interaction functions that can be associated with each of the interaction matrix group using notation from Eq. [4] and [6]. However, the selection of the interaction function class might change depending on case and is optimized against data in the most general case. Note, that some of the interaction terms are zero by definition—they are indicated with 0 at the proper locations in the general form of the interaction matrix presented in FIG. 7. The nulled interaction terms are primarily related with algebraic definition of total cell surface, with definition of inaccessible toxicant, as well as with diagonal relocation-block related terms. More than one interaction term per block indicates that the form of terms differs between the block diagonal and/or upper and/or lower triangle (also indicated by different filling color behind). Interaction function dependence notation involves observable together with 1 or 3 indexes (in line with definitions from Eq. [6] and [7]): 1^{st} index is always index of observable i or j, where i always denotes the row index in a block row, j always denotes the column index in a block-column, 2^{nd} index is possible/expected derivative o₁ and 3^{rd} index is possible/expected derivative o₂; if the 2^{nd} and 3^{rd} indexes are omitted or denoted by x, they can take any of the possible integer values and are allowed to be determined throughout the parameterization procedure. Multiple occurrences of interaction functions that depend on the compartment sizes (F(Ci)) appear in different forms (with different indexes) in the same interaction term due to transformation of a quotient-dependent interaction function as explained before (for example, one occurrence acts in terms of compartment-associated normalization from concentration-like dependence and another from other compartment-dependent interaction function)—in such cases derivatives o₁ and o₂ are strictly defined—they correspond to the transformation rule and are not free to be determined by the experiments.

In case of less complex interactions between the observables (events) are detected or less complex biological system is tested, the interaction matrix may be further simplified by omitting or excluding additional parts or designating their value as zero (0). Such a case is further discussed Example 1 and FIG. 16.

### g) Base Function and Super Equation Formation

The next step is to transform the system of differential equations into a set of terms, i.e., linear products of interaction matrix element power parameters and base functions (that are sums of products of interaction functions) to prepare the system for automatic parametrization—determination of power parameters. To implement the latter, every derivative value of a selected observable dqᵢ/dt (defined through the corresponding differential equation—left side of Eq. [3]) is first perceived as a linear combination of products of interaction functions weighted by the parameters p_{i,j} (combined Eq. [3] and [4]).

$$\frac{d}{dt} {q}_{i} = {\sum }_{j} ( {p}_{i , j} {\prod }_{k ∈ i , {j}^{*}} {F}_{o ( k )} ( {q}_{k} ) ) \qquad [ 11 ]$$

It can be seen in examples of the interaction matrix terms (see functions listed in FIG. 7) that some blocks mix the interaction terms and some parameters p_{i,j} appear in more than one interaction term (see function sets presented in Example 1), making these terms linearly dependent. When determining the value of such a parameter with highest sensitivity, Eq. [11] must be rewritten as a linear combination of linearly independent expressions

$$\frac{d}{dt} {q}_{i} = {\sum }_{j} ( {p}_{i , j} {\sum }_{l} ( {\prod }_{k ∈ {i}_{l} , {j}_{l}^{*}} {F}_{o ( k )} ( {q}_{k} ) ) ) = {\sum }_{j} ( {p}_{i , j} {B}_{i , j} ( \{ {q}_{b} \} ) ) \qquad [ 12 ]$$

where B_{i,j} (sum of products of interaction functions) associated with p_{i,j} becomes a base function dependent on a union of observables {q_{b}} that appear in the interaction functions inside the black-written sum of Eq. [12].

To be as sensitive as possible, determination of parameters via Eq. [12] should be done in subgroups of equations in which only a subgroup of parameters appears, while other parameters do not (they appear only in the complement of this particular subgroup of equations). This means that other parameters can be independently determined through other (subgroups of) equations. Symbolically, each such subgroup of equations will be referred to as a super-equation.

When solving such super-equation (system of parametrically coupled algebraic equations), also the parameters and base functions need to be collected (mathematically speaking replaced by union of parameters and union of base functions associated with the equations coupled in one super-equation). Symbolically, this will be referred to as a super parameter set and a super base set.

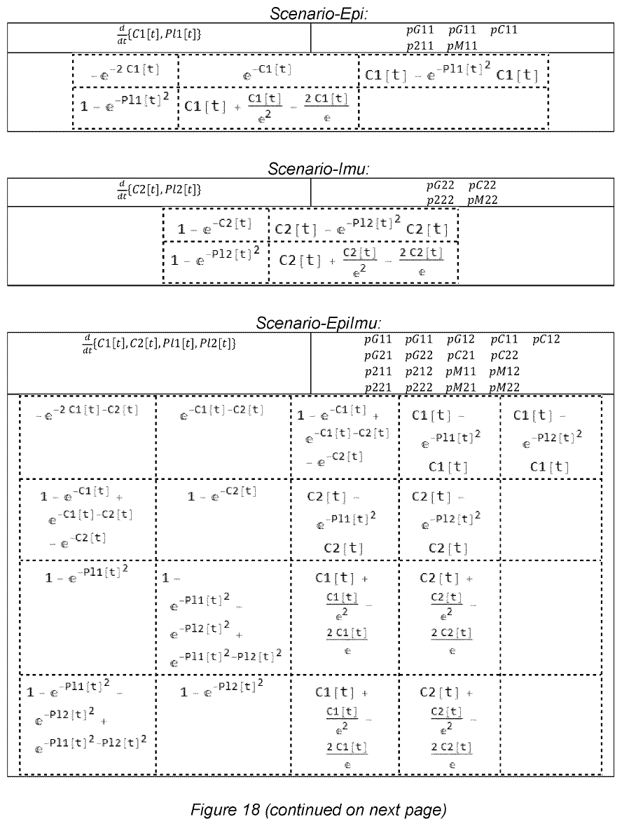

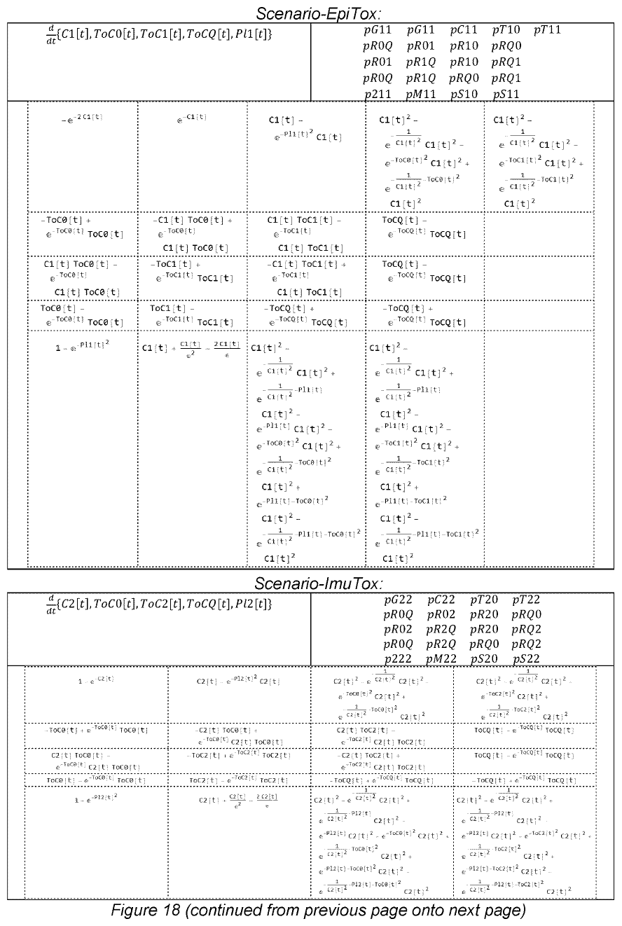

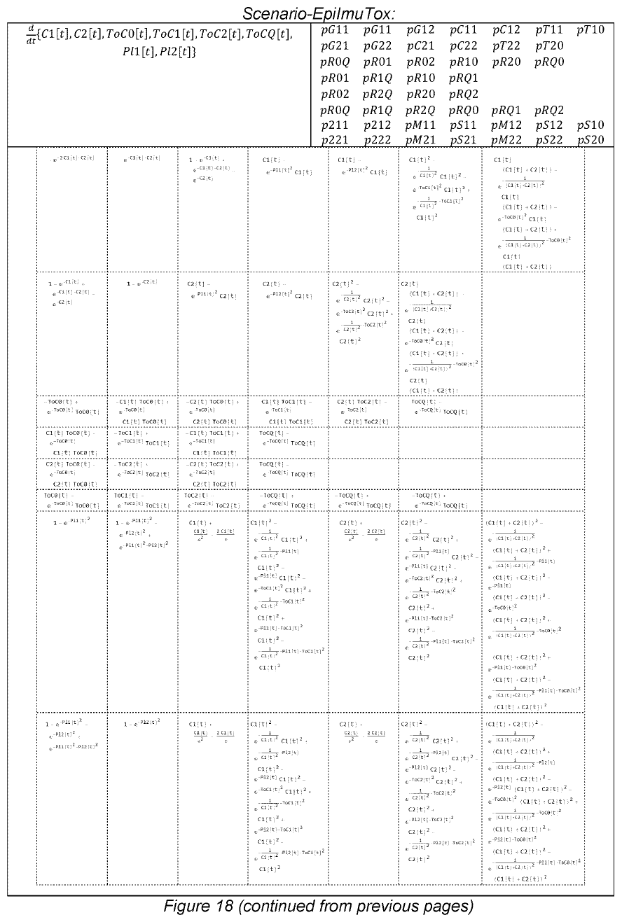

Examples of super base function sets and the corresponding parameters are shown in Example 1 in FIG. 18.

Note that the number of algebraic equations used in the determination of a super parameters set (group of parameters through one super-equation) is defined by the following product:

$${N}_{algebraic equations} = {N}_{ROI} \times {N}_{time points} \times {N}_{scenaria} \times {N}_{cupled equations} \qquad [ 13 ]$$

### h) Parameterization of the Interaction Terms

Taking into account a typical experimental dataset, which involves data from around 10 ROIs over at least 15 time points for each of the 6 scenaria, and 1-4 coupled equations per one super equation, the number of algebraic equations per single task exceeds 100 or even 400 (e.g. in case of parameters that are associated with relocation of the toxicant). Because the number of parameters that appear in such algebraic systems never reach such a high number, the algebraic systems are always overdetermined and need to be solved with maximum likelihood or least squares-driven optimization routines, singular value decomposition or similar methods.

Here we show how this system is solved employing Singular value decomposition (SVD) due to its simplicity and computational efficiency. This, however, does not preclude the implementation of other routines, such as linear or nonlinear optimization routines. While SVD is the most computationally efficient, optimization routines enable more straightforward implementation of constrains of power parameters to certain ranges, e.g., to positive values (see below).

Independently on the method chosen, the optimization problem is defined first. For this purpose, the data on observables' values and their derivatives are first used to construct the matrix M (not to be confused with IM) from the right-hand sides of the set of Eq. [12], where:
each row represents the super equation with many terms, where {t, {qᵢ}, {dqᵢ/dt}} are substituted by their fitted values according to Eq. [2] that correspond to the particular combination of ROI, time point and scenario and
each column represents the value of one of the base functions that is associated with the p_{i,j} (i.e. with the column) according to Eq. and is a member of a super base set.

The number of columns equals the number of parameters that can be defined within one super equation, while the number of rows equals the number of algebraic equations in one super equation defined by Eq. [13].

On the other hand, the left-hand sides of the set of Eq. [12], i.e. all the derivatives of observables taken from {t, {qᵢ}, {dqᵢ/dt}} for each combination of ROI, time point and scenario, are contained in the vector Y. As by Eq. [12], Y equals the product of the matrix M with the vector of parameters P (Eq. [14]):

$$Y = M . P \qquad [ 14 ]$$

$$[ \begin{matrix}{\frac{d}{dt} \vert}_{{t}_{1} , {ROI}_{1}} \\ {\frac{d}{dt} {q}_{1} \vert}_{{t}_{2} , {ROI}_{1}} \\ ⋮ \\ {\frac{d}{dt} {q}_{1} \vert}_{{t}_{1} , {ROI}_{2}} \\ {\frac{d}{dt} {q}_{1} \vert}_{{t}_{2} , {ROI}_{2}} \\ ⋮ \\ {\frac{d}{dt} {q}_{2} \vert}_{{t}_{1} , {ROI}_{1}} \\ {\frac{d}{dt} {q}_{2} \vert}_{{t}_{2} , {ROI}_{1}} \\ ⋮\end{matrix} ] = [ \begin{matrix}{{B}_{1 ; 1 , 1} ( \{ {q}_{b} \} ) \vert}_{{t}_{1} , {ROI}_{1}} & {{B}_{1 ; 1 , 2} ( \{ {q}_{b} \} ) \vert}_{{t}_{1} , {ROI}_{1}} & … & & & \\ {{B}_{1 ; 1 , 1} ( \{ {q}_{b} \} ) \vert}_{{t}_{2} , {ROI}_{1}} & {{B}_{1 ; 1 , 2} ( \{ {q}_{b} \} ) \vert}_{{t}_{2} , {ROI}_{1}} & … & & & \\ ⋮ & ⋮ & ⋱ & & & \\ {{B}_{1 ; 1 , 1} ( \{ {q}_{b} \} ) \vert}_{{t}_{1} , {ROI}_{2}} & {{B}_{1 ; 1 , 2} ( \{ {q}_{b} \} ) \vert}_{{t}_{1} , {ROI}_{2}} & … & & & \\ {{B}_{1 ; 1 , 1} ( \{ {q}_{b} \} ) \vert}_{{t}_{2} , {ROI}_{2}} & {{B}_{1 ; 1 , 2} ( \{ {q}_{b} \} ) \vert}_{{t}_{2} , {ROI}_{2}} & … & & & \\ ⋮ & ⋮ & ⋱ & & & \\ & & & {{B}_{2 ; 1 , 1} ( \{ {q}_{b} \} ) \vert}_{{t}_{1} , {ROI}_{1}} & {{B}_{2 ; 1 , 2} ( \{ {q}_{b} \} ) \vert}_{{t}_{1} , {ROI}_{1}} & … \\ & & & {{B}_{2 ; 1 , 1} ( \{ {q}_{b} \} ) \vert}_{{t}_{2} , {ROI}_{1}} & {{B}_{2 ; 1 , 2} ( \{ {q}_{b} \} ) \vert}_{{t}_{2} , {ROI}_{1}} & … \\ & & & ⋮ & ⋮ & ⋱\end{matrix} ] ⊗ [ \begin{matrix}{par}_{1 , 1} \\ {par}_{1 , 2} \\ ⋮ \\ {par}_{2 , 1} \\ {par}_{2 , 2} \\ ⋮\end{matrix} ]$$

Optimization problem can now be defined as minimization of

$${‖ M . P - Y ‖}_{2} \qquad [ 15 ]$$

by changing the elements of P (i.e. the searched-for values of power parameters).

When employing the SVD method, the matrix M (real, rectangular) can be expressed as a matrix product:

$$M = U . W . {V}^{T} \qquad [ 16 ]$$

where
W is a diagonal matrix with values wᵢᵢ being the singular values of the matrix M
U and V are column orthonormal matrices.

When minimizing by means of SVD, the parameter vector P can be expressed as

$$P = V . ( {U}^{T} . Y ) / w \qquad [ 17 ]$$

where U and V are defined via Eq. [16], w is the vector of the diagonal elements of W, also defined via Eq. [16], and the division by a vector represents element-wise division.

To keep the parameterization as reliable and as meaningful as possible and prevent ill-posed parameterization, two adjustments have been implemented.

The first is used only in case of SVD, while the second one is implemented also, when the parameterization is done with other optimization routines:

When employing SVD in minimizing [15], the rank of SVD (i.e. the dimensions of W) is preferably minimized as follows: SVD is first run with maximal rank to determine the vector w with all the singular values. Then, the rank of SVD is reduced to the number of singular values that exceed some low relative threshold, e.g. 5% of the maximal singular value, and SVD is run again. This effectively reduces the rank of SVD to a minimum needed to satisfactorily describe the noisy experimental data.

On the other hand, regardless of the chosen minimization/optimization method (SVD or any other), ill-posed parameterization is prevented also by restricting the selected parameters to positive values. As per the physical meaning of some of the equations, some parameter values must take positive values, such as those describing mass-retaining translocation of the toxicant (central block of the central block row). Because other elements in the central block row of the interaction matrix in principle allows reduction or generation of toxicant's amount (due to biodegradation, aggregation, adsorption, inactivation, passivation, and even biosynthesis), some of the interaction terms might compensate each other during determination of parameters, potentially leading to wrong parameterization that later on results in oscillations in final observables' time propagation. Because such ill parameterization often originates in the wrong sign of the parameters, it is useful to maintain their positive values. If a positive defined parameter (for example relocation parameters in the central block of the interaction matrix) appears to be negative after the first run adjustment 1), it is locked to 0 and determination of parameters with SVD is run again. In practice, the entire determination procedure becomes stable in less than 3 iterations even with an extremely complex interaction matrix.

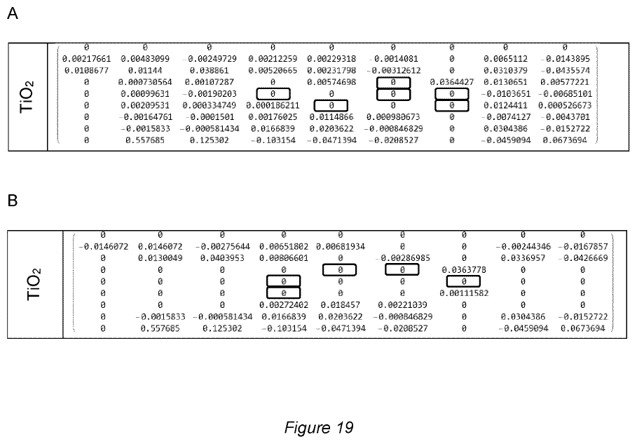

In Example 1, the parameters, which are affected by the above approach to prevent ill-parameterization by restricting the values to positively defined values, are indicated with boxes in FIG. 19.

### i) Scenario-Dependent Fit to In Vitro Experiments

Parameterization of the interaction matrix corresponds to determination of the observables' derivatives. Observables' values, however, are determined from their corresponding derivative values using the corresponding initial conditions.

With the entire interaction matrix being parameterized (all the terms in the differential equations become numerically defined), (early) evolution of the observables are defined for each of the scenaria using initial conditions as follows: Initial conditions might equal the experimental initial conditions, when mathematical model is used to describe early evolution of in vitro model. On the other side, initial conditions might also differ from the experimental initial conditions, when mathematical model is used in the final step to time-propagate observables to mimic real in vivo situation.

The easiest way of determining the initial values of the observables is taking the mean of the earliest data points for each corresponding observable (averaging ROIs if applicable). However, initial conditions might also be taken from the in vivo system (if applicable or measurable) or from the available data resources.

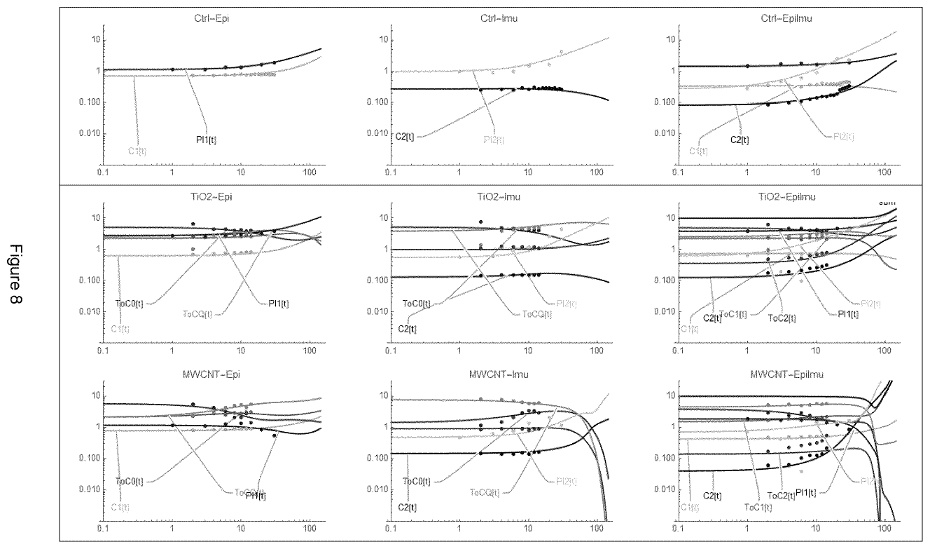

An exemplary fit to the early time evolution of all the observables monitored within the in vitro model, which corresponds to the Example 1, is shown in FIG. 8. Here we present the measured time-evolution of all the observables (dots) together with their in silico fit (lines) monitored within a lung in vitro model (epithelial cell+lung macrophages) for all the scenaria (3 control+3 exposure-related) for two materials to confirm quality of the translation of in vitro observed dynamics to in silico parameterization.

### j) Mechanism Presentation

The determined parameterization defines the contributions of interaction matrix elements (i.e., time derivatives in the systems of differential equations), from which the mechanism of action can be presented if desired. Because the disclosed method determines how pairs of observables effect each-others' values change in time, where the magnitude of the effects is parameterized by the power parameters in the interaction matrix, the generalized interaction matrix contains a large number of such parameters, which are thus difficult to inspect quickly.

To allow fast inspection of the parameterization of the interaction matrix and easier identification of the key mode-of-actions in individual-material exposures, there is optionally provided a method of mechanism presentation.

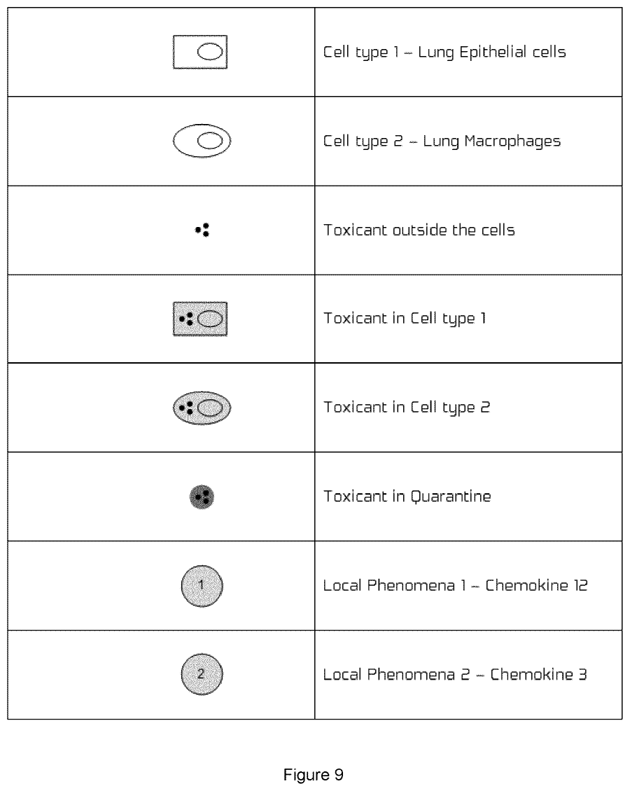

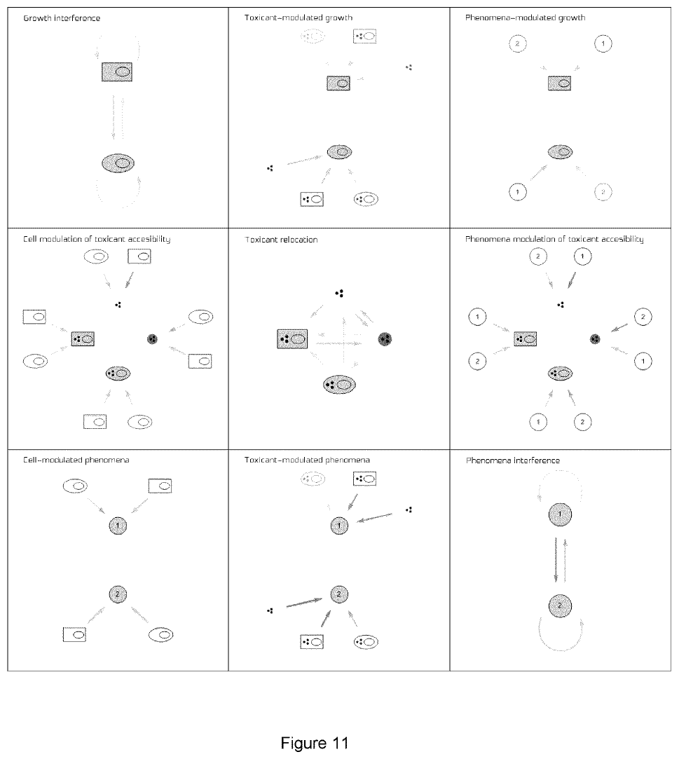

With this step, presentation concept of the key mode-of-actions is provided to allow efficient comparison of the mechanistic evaluation between materials or individual-material exposures. One mechanism presentation concept (see FIGS. 9, 10 and 11) is defined as follows:
Quantities are depicted by the pictograms as presented in FIG. 9,
Effects are depicted by arrows with triple encoding (see example in FIGS. 10 and 11, which belongs to determined mode of action for the exposure of Example 1 and 2)—larger effects are encoded by thicker non-transparent arrows with saturated colour, smaller effects are encoded by thinner almost transparent arrows with less saturated colour, insignificant effect are barely seen due to almost pure transparency and grey colour; the positive and negative sign of effect is encoded by green and magenta colour, respectively,
Quantities that are affected (those on the left side of differential equations) lie in the inner circle with their pictograms being grey filled, while the quantities that cause the effect lie on the outer circle with white-filled pictograms
Self-stimulating and self-inhibitory effects are depicted with semi-circular arrows that originate and target the same pictogram (of the quantity to which the self-stimulating or self-inhibitory effect refers).

FIG. 9 thus shows the graphical symbols (pictograms) that are used to illustrate the elements of a mechanistic report. The particular case shown corresponds to the coculture of 2 cell lines (lung epithelial cells and lung macrophages) and 2 phenomena being chemokine 12 (CXCL12) and 3 (CCL3) excreted by epithelial cells and macrophages, respectively):

According to the scenario concept, it is obvious that some of the blocks determine the function of the biological processor per se (biological control), while other blocks determine the response of the processor to the exposure to the agent (toxicant).

### k) Dose Biodistribution Characterization

Because animal-based disease prediction is based on the long-lasting observation of end-points (symptoms, adverse outcomes, disease, etc.), determination of dose-related information such as no-observable-adverse-effect-level (NOAEL dose) and least-observable-adverse-effect-level (LOAEL dose), is even longer and more expensive.

On the other hand, the presently disclosed methods in which disease prediction is realized through in vitro monitoring of early-events coupled with in silico time propagation is obviously much faster and consequently much cheaper than original animal-based testing. Firstly, it is 10-30-times faster in observing the prediction-required early key events (in comparison with the time needed to observe prediction-needed end-points in animal-based testing). And secondly, the local bio-relevant/bio-distributed doses can be derived directly from images of the exposed in vitro models in terms of dose-distribution histograms within a single exposure experiment (on the contrary to the animal-based experiments, where each of different doses requires its own exposure experiment and subgroup of animals to be associated with disease triggering efficiency).

Dose biodistribution characterization (derived within in vitro—in silico approach) may rely on an experimental fact that under real conditions no tested agent (toxicant/material/chemical/etc) can be delivered evenly into the biological system, independently on the way of delivery. Because early events on a cellular and subcellular levels are monitored and colocalized with distribution of the tested substance (toxicant), the method can directly analyse the dose response from the image-based dose distribution.

For that purpose, dose biodistribution is assessed as followed:
the images from the channel that delivers or is related with the information about the toxicant are segmented (by one of the available algorithms of the image segmentation) to create a list of agent (toxicant)-related objects,
optionally, the list of the segmented objects can be additional modified by excluding the objects based on objects' size or other descriptors, where the latter can be calculated via one of the available algorithms taking into intensity, dimension, aspect ratio, edge length/surface, skeleton information, mass or cross-section size, etc.,
optionally, the object might be further split into surface of the object (accessible agent) and bulk of the object (inaccessible agent) if required or assumed by the model,
for each of the segmented objects in the list, its dose equivalent is calculated as:
   dose in terms of mass or surface area of the agent (summing the intensity of the pixels within the segmented object if the intensities do not depend on labelling/identification process-valid for example for Raman-based determination)
   model-corrected dose in terms of mass or surface area of the agent (summing the number of pixels within the segmented object if the intensities do depend on labelling/identification process-valid for example for fluorescence-based or scattering-based determination; the model needs an advanced calibration for density of the information, scattering intensity, etc.)
   dose in terms of interacting dose (summing only the intensity of those pixels that are in contact with the biological system of interest; might be the surface of the object only, or even only part of the surface, for example the surface which is in contact/colocalized with membrane, proteins, RNA, etc.)
from the list of objects and their associated doses, a dose histogram is created showing the number of objects that exhibit certain dose (bin, subrange); wherein number of dose bins are defined based on the total number of objects in the analysis.

Because the effect propagation depends on the local dose, the multi-ROI images are used to determine the histogram of dose distribution.

Various combination of dose concepts is used in calculation of prediction:
For the barrier-like organs (with large surface-to-volume; such as lungs or liver) surface (of materials)-to-surface (of cells) local dose is used for materials and mass (of compound)-to-surface (of cells) for insoluble compounds,
For voluminous organs (thick with small surface-to-volume; such as muscle or brain) surface (of materials)-to-mass (of organ/body) is used for materials and mass (of compounds)-to-mass (of organ/body) for soluble compounds;

Different dose concepts is used in delivery of final reports (because of the exposure related issues):
For the barrier-like organs mass (of compound)-to-surface (of lungs) for insoluble compounds, later recalculated to mass (of compound)-to-time (of exposure) (because these organs are likely to be multiple-exposed, which is normally related to time of exposure);
For voluminous organs mass (of compounds)-to-mass (of organ/body) for soluble compounds, always recalculated to mass (of compound)-to-time (of exposure).

The above-described calculation of dose biodistribution is also shown in FIG. 12.

Optionally, the observed dose (D) (for each bin in the delivered histogram) can be related to a dose rate (dD/dt), which is frequently measured in a real environment, and the critical exposure time t_{c} can be derived

$${t}_{c} = D / ( dD / dt ) \qquad [ 18 ]$$

needed for a dose D to accumulate in that environment or organism at a measured rate dD/dt.

### l) Long-Time Propagation of Observables-AO Prediction

After in vitro-based parameterization is fully derived, the final prediction of the adverse outcome development is calculated as a long-term time propagation of the early observables' propagation after the exposure to the material/chemical/drug/agents of interest.

Because in a real organism a long-term effect inherently involves a systemic response beside the local tissue response, the adverse outcome prediction should involve a coupling between the local tissue evolution, determined and parameterized through the in vitro model early evolution monitoring (even the most complex one), and the systemic effect, which covers interaction of the in vivo response of the organism with local tissue.

This coupling finally translates the evolution of the early local events into the response, that can propagate the effect of the organism as well making the final adverse outcome/symptoms/disease prediction much more relevant. Such an approach employs complex knowledge from AOP or mode-of-action database, which collects the events that critically define the evolution of adverse outcomes in terms of complex response of organisms.

In the present methods, this knowledge is implemented by expanding the interaction matrix (see dotted region indicated in FIG. 13). Beside the already parameterized part of the interaction matrix defined through in vitro system, the expanded interaction matrix thus implements additional information based on AOP representing by local-to-system coupling (horizontal part on the bottom) and system-to-local coupling (vertical part on the right). The additional part of the interaction matrix provides the mean to couple local events to systemic events and vice versa. By definition, the former occurs within the in vitro model and the latter outside the in vitro model. For example, when a cytokine is released within the in vitro model, it is considered as a local event. However, when it leaks from a local tissue into a blood stream, it is considered as a systemic event outside in vitro model, that would be used to model lung alveolar epithelial. Because it is related to local event, this method models it under local-to-system coupling region of the interaction matrix. Note that, if an in vitro model includes vascular endothelial barrier as well, such a leak would remain local event. On the other hand, systemically released signalling molecules might call new immune cells to enter alveolar tissue (for example monocytes or PMN cells that would enter the lung from blood as a response to local immune cell attractant release). Such an event would be considered systemic, which however directly affect the local observables (number of immune cells in the local tissue). In the presently described method it is thus covered by system-to-local coupling. Logically, the expanded part of the interaction matrix relies on the in vivo-relevant observables that are related to adverse outcome prediction and are defined on the beginning of this method.

The interaction terms of the expanded part of the interaction matrix can have arbitrary forms and can depend on any combination of observables from the inside of the in vitro model, as well as of observables from outside the in vitro model.

Validation of the method may be done by comparing the obtained results with previously published data.

The described methods provide a solution for safety and/or toxicity prediction of possible toxicants such as materials, chemicals, medicines, vaccines and similar substances and agents. It can predict the adverse outcome ahead of time with regards to adverse outcome evolution in vivo, within animal-based testing. It is thus (much) faster, more cost-efficient and readily applicable to various experimental set-ups.

### EXAMPLES

The following examples are given to illustrate various embodiments of the disclosed methods and their use, but should not be viewed as limiting.

### Example 1—Comparison of the Method and Results Published by Kokot et al (2020) on Chronic Inflammation Prediction for TiO₂ Nanotubes

The first example aims to illustrate the ability of this method to independently determine the early mode-of-action and transform the latter into safety assessment exclusively with monitoring of in vitro models. This example is related to the long-term toxicity prediction for the material (TiO₂ nanotubes), for which mechanistic research was published in Kokot et al. Adv. Mater. 2020 (so the mechanism revealed here can directly be compared to the published one) and in vivo data is available for validation (so the prediction can directly be compared to the real in vivo data).

This example exemplifies all the steps of the method according to an embodiment of the present disclosure (FIG. 2).

### a) Associative Identification of Relevant Observables:

Based on the published in vivo data related to inhalation of TiO₂ nanotubes (H2020 project SmartNanoTox reports and publications, such as Adv Mater. 2020 doi: 10.1002/adma.202003913, Toxicol. Appl. Pharmacol. 2020 doi: 10.1016/j.taap.2019.114830), the in vivo-relevant observables for chronic inflammation prediction are:
the surface of epithelial cell constituting the alveolar surface being the first candidate to be affected during the exposure,
the number of macrophages, because after the exposed lungs monocytes enter the lungs and differentiate into macrophages replacing dead residential macrophages, and polymorphonuclear (PMN) cells enter the lungs and their influx strongly correlate with influx of the monocytes,
the surface of quarantined material, because the process of quarantining is a direct consequence of the attractive interaction between many materials and many biological molecules (confirmed by MD studies and high-resolution in vitro as well as in vivo microscopy)

In addition, the in vivo relevant observables in this case (confirmed by the transcriptomics study of the 50.000 gene expression for several material exposure) are also the following cytokines:
CCL 3—Macrophage inflammatory protein (MIP)-1α/CCL3—an inflammatory chemokine produced by cells during infection or inflammation, detected via Mouse CCL3 ELISA kit (Proteintech, KE10023),
Cxcl 12/CXCL12—a constitutive chemokine involved in the lung, brain, and joint inflammation, detected via Mouse CXCL12 ELISA kit (Proteintech, KE10049),

CCL 4/CCL4, also known as Macrophage inflammatory protein-1β (MIP-1β)—a CC chemokine with specificity for CCR5 receptors, being a chemoattractant for natural killer cells, monocytes, and a variety of other immune cells, detected via Mouse CCL4 ELISA kit (Proteintech, KE10030)

### b) Preparation of an In Vitro Model

To replicate the lung alveolar tissue, which was exposed to sub-3-micron-sized particles/substance that are not cleared out by mucociliary escalator (dimensions of TiO₂ nanotubes are 10 nm in diameter and 100-200 nm in length), a lung-mimicking in vitro model was comprised from lung epithelial cells (their surface is depicted with observable Compartment 1 (Epi)—C1) and lung macrophages (their surface is depicted with observable Compartment 2 (Imu)—C2).

### Lung Mimicking In Vitro Model Comprises:

| | |
|---|---|
| LA-4 | Murine respiratory alveolar epithelial cells |
| | commercially available from the American |
| | Type Culture Collection (ATCC, www.atcc.org |
| | or www.lgcstandardsatcc.org). This cell line |
| | is adherent and is cultured in Ham's |
| | F-12K medium supplemented with 2 mM I - |
| | glutamine (Gibco), 50 U/ml penicillin, 50 |
| | mg/ml streptomycin, and 15% FBS, at 37° C. |
| | in a humidified 5% CO₂ environment. |
| MH-S | Murine alveolar macrophage cell line |
| | commercially available from the American Type |
| | Culture Collection (ATCC, www.atcc.org or |
| | www.lgcstandardsatcc.org). This cell line is |
| | partially adherent and is cultured RPMI-1640 |
| | Medium, supplemented with 2-mercaptoethanol |
| | to a final concentration of 0.05 mM, 50 U/ml |
| | penicillin, 50 mg/ml streptomycin and 10% FBS, |
| | at 37° C. in a humidified 5% CO₂ environment. |
| Coculture | Both cell cultures are grown together in a |
| LA-4 & MH-S | ratio of 1 MH-S cell to 10 LA-4 cells |
| | in a 1:1 mixed medium. |

Cell lines are weekly checked for morphological changes, viability, and metabolic activity. Only cells that are in the range of quality control are entering into the experiments.

### c) Scenaria-Based Time-Lapse Acquisition

Methods to acquire high-throughput in vitro time-lapse data:
fluorescence confocal microscopy with partitioning-based labeling and cell-transfection-based labeling with excitation/detection bands being: 488/500−550+561/570−630+640/650−710 nm,
confocal back-scattering (dark-field) microscopy with excitation/detection at 488/488 nm,
ELISA for detection of CCl3, CCl4, and Cxcl12.

With additional supportive measurement used for identification of observables:
Helium-ion microscopy (HIM),
Scanning electron microscopy (SEM),
Raman microscopy,
Transcriptomics,
Proteomics of coronome.
Number of time points: 14 for microscopy and 5 for ELISA
Number of ROI (range-of-interest) in each scenaria: 10
Number of slices per z-stack: 4 (height difference of 3 microns)
Total duration of acquisition: 30 h

The observables used in this prediction:

| | | |
|---|---|---|
| Cell | C0 | Total surface area of (all) cells |
| surface | C1 | Surface area of epithelial cells |
| areas | C2 | Surface area of immune cells |
| (comaprtments) | | |
| Amounts | ToC0 | Amount of toxicant outside of (all) cells |
| of | ToC1 | Amount of toxicant in epithelial cells |
| toxicants | ToC2 | Amount of toxicant in immune cells |
| | ToCQ | Amount of toxicant in quarantine |
| | | (physical + biological) |
| Phenomena | Pl1 | Concentration of expressed Cxcl12 |
| | PI2 | Concentration of expressed Ccl3 |

Number of Scenaria: 6
Scenaria description:

| Scenaria | | Epi | Imu | EpiImu | EpiTox | ImuTox | EpiImuTox |
|---|---|---|---|---|---|---|---|
| Surface | C0 | 1 | 1 | 1 | 1 | 1 | 1 |
| areas | C1 | 1 | 0 | 1 | 1 | 0 | 1 |
| (comaprtm [symbol] | C2 | 0 | 1 | 1 | 0 | 1 | 1 |
| Amounts | ToC0 | 0 | 0 | 0 | 1 | 1 | 1 |
| of | ToC1 | 0 | 0 | 0 | 1 | 0 | 1 |
| toxicants | ToC2 | 0 | 0 | 0 | 0 | 1 | 1 |
| | ToCQ | 0 | 0 | 0 | 1 | 1 | 1 |
| Phenomena | Pl1 | 1 | 0 | 1 | 1 | 0 | 1 |
| | Pl2 | 0 | 1 | 1 | 0 | 1 | 1 |

[symbol]

indicates data missing or illegible when filed

Where applicable (scenaria with exposure: EpiTox, ImuTox, EpilmuTox), the exposure relevant data are as follows:

| | |
|---|---|
| Vehicle used | 1 mM bicarbonate buffer, pH 10 |
| Explanation | The buffer has to have low osmolarity and high pH, |
| of vehicle | because of the charge screening of the active surface of |
| | the material. |
| Preparation | 6 mg of material were diluted in 1 mM bicarbonate buffer |
| protocol | to obtain 1 mg/ml concentration. The material was |
| | dispersed by sonification on an ice bath for 15 min run |
| | time with 5 s on - 5 s off regime (30 min total time). The |
| | power was set at 20-30 W (amplitude 70). |
| Dosage | 1 mg/ml; this dose has been recalculated from the 10:1 |
| | surface dose in terms of the nanotube-surface-to-lung- |
| | surface dose, which is near the in vivo dose, for which |
| | least-observable effect level (LOAEL) was detected |
| | (Toxicol. Appl. Pharmacol. 2020 doi: |
| | 10.1016/j.taap.2019.114830) |
| Explanation | We calculated the appropriate dosage of the |
| for dosage | nanomaterial based on the average surface of the |
| | lungs. |

The solution of the tested material was added to the in vitro model dropwise to cover the whole surface of the in vitro model. The volume applied to cells never exceeds 10% of the medium volume. The total duration of exposure is 30 h, all the tests were performed in different time points within the 30 h. Different interaction dosages are calculated from the different regions of interest (ROI).

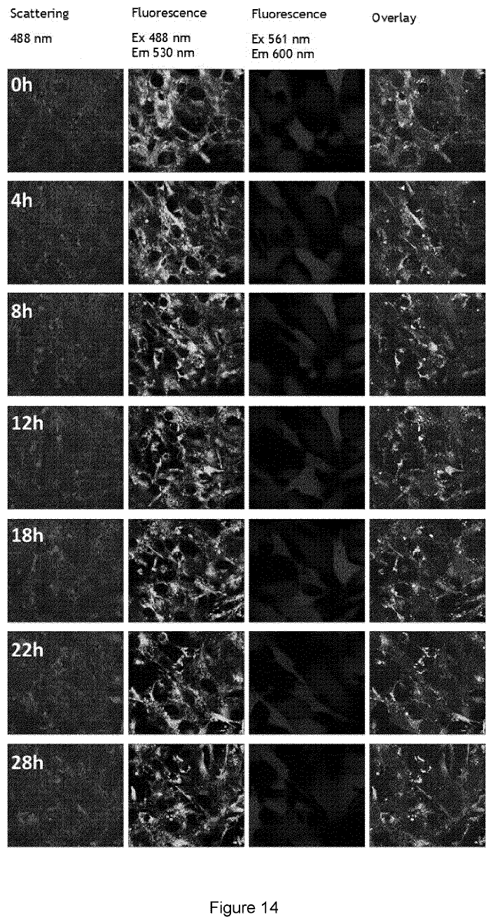

Representative example of the acquired images of this experiment (fluorescence and backscattered microscopy) is presented in FIG. 14. Here we show part of the images from a time-lapsed 3-channel (two fluorescence and one back-scattered) monitoring of TiO₂-exposed in vitro lung model. As it can be seen, these images and the selection of preceding acquisition methods allow identification of objects and compartments, their segmentation, further quantification, and colocalization of events.

Fluorescent probes used in this particular experiment were:
CellMask Orange to label all cells,
transfected cell lines with GFP to label epithelial cells only.

Nanomaterial was tracked by confocal back-scattered microscopy.

### d) Quantification of Observables by Image Analysis

Epithelial cells mask (C1) was generated directly from transfected cell lines channel images by binarization (thresholding above 3 times of average background noise at positions with no cells). To exclude the error from cell overgrowth, masks from different z-slices have been down-projected (final masks pixel value equals to union of individual masks pixel values).

Immune cells mask (C2) was generated as a difference from a mask corresponding to CellMask Orange channel and a mask corresponding to transfected cell lines channel again by binarization (thresholding). To exclude the error from cell overgrowth, masks from different z-slices have been down-projected before subtraction (final masks pixel value equals to union of individual masks pixel values).

Particle mask has been derived from back-scattered images at lower z-slice. The images of the upper z-slices have been used to derive mask for material outside cells (ToC0). Particle mask have been segmented to identify objects greater than 3 pixels in diameter. List of objects has been filtered according to object size, excluding the objects smaller than 1 micron. Then new material mask has been constructed to take into account only masks of larger objects with border of 3 pixels being excluded. This has been denoted as ToCQ masks (quarantine).

After subtracting ToCQ mask from original material mask, the remaining mask (ToC) has been used to derive material mask inside epithelial (ToC1) and immune cells (ToC2) by multiplying the ToC mask with the corresponding cell masks C1 and C2 from previous steps.

The intensity of the material within particular compartment has been derived by summing up the intensity of the pixels in the corresponding masks ToC0, ToC1, ToC2, ToCQ.

The employed process is depicted in FIG. 5.

### e) Generation of Time Series and Time Derivatives

The entire set of experimental data in the form of {{tⱼ}, {qᵢ (tⱼ)}, {dqᵢ/dt(tⱼ)}} are transformed into {{tⱼ}, {q_{i-f}(tⱼ)}, {dq_{i-f}/dt(tⱼ)}} by fitting {qᵢ(tⱼ)} to {q_{i-f}(tⱼ)} using Eq. 1. Original set of experimental data can be seen as dots on FIG. 5.

### f) Construction of an Interaction Matrix

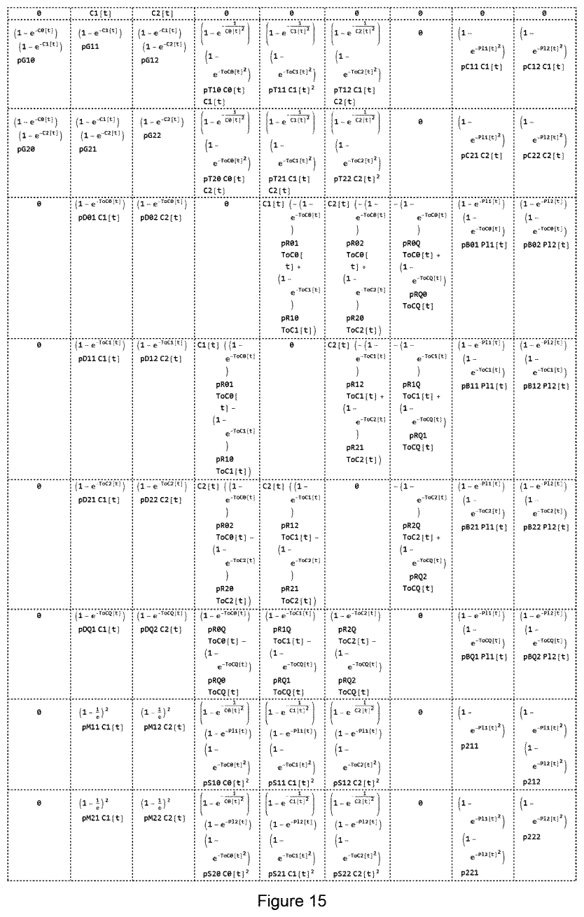

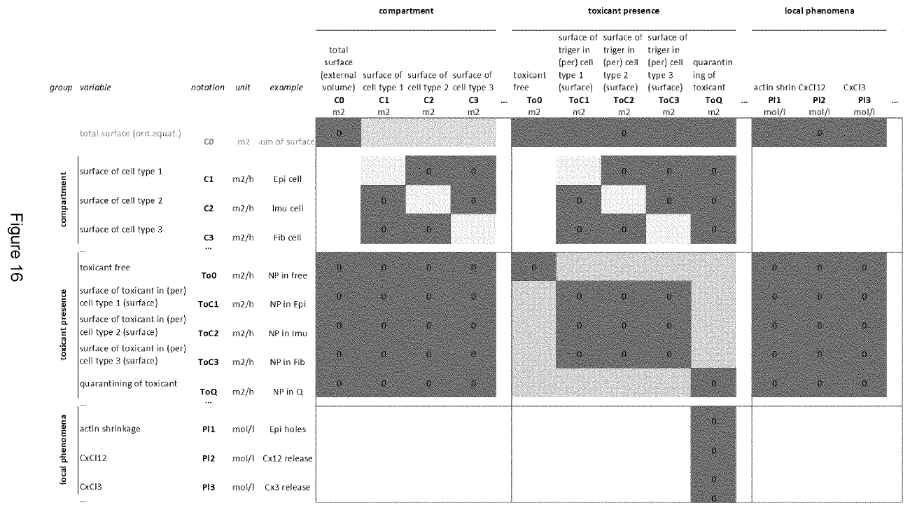

To define the time propagation of vector of observables (definition of observables in Step c) from above, definition of time propagation in Eq. 10), interaction matrix elements are constructed (definition in Eq. 4 and FIG. 7) through individual interaction function (definitions in FIGS. 6 and 7 using list of possible function defined through Eq. 6 and 7). Entire automatically constructed interaction matrix used in prediction of TiO₂ nanotubes is presented in FIG. 15 (due to excessive size, elements are wrapped and gridlines are added; the power parameters p_{x,ij} are already named after their function and block location within the 3×3 blocks of the interaction matrix).

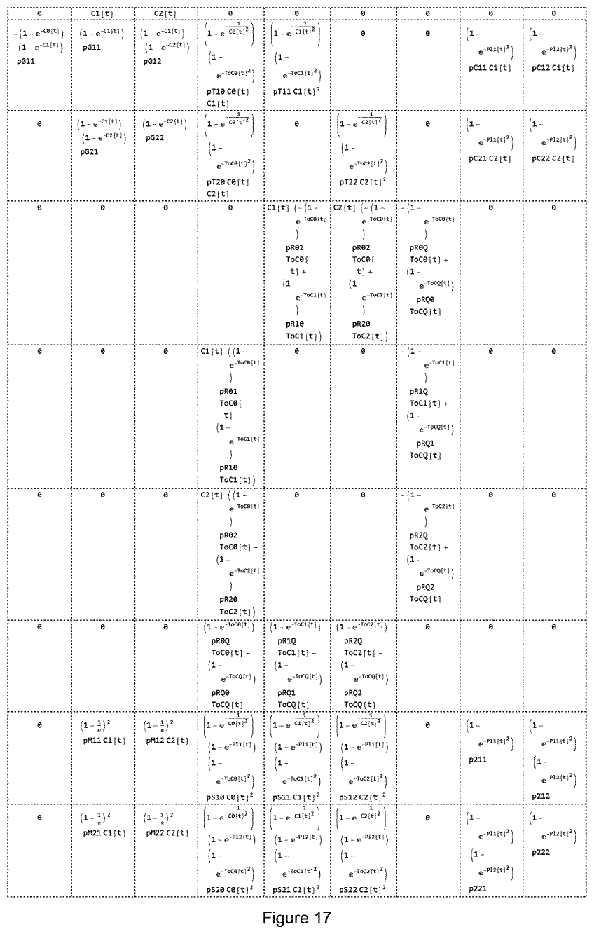

Because TiO₂ nanotubes are practically unsolvable, interaction matrix can be even further simplified—mostly for presentation purposes (see FIG. 17)—but can still be used in prediction of TiO₂ nanotubes. In this case, blocks D and B are nulled and some other small simplifications are implemented as well (see FIG. 16).

### g) Construction of Base Functions and Super-Equations

Base functions and super-equations were created by using the above-described Eq. 12.

Here, the example of base function list (together with the corresponding time-derivatives of observables and power parameters), sorted by equations (in rows) are shown in FIG. 18. Note, that this image presents the base function list for simplified version of interaction matrix—the base functions of the generalize interaction matrix would be too excessive-sized to be presented here.

### h) Parameterization of Interaction Terms

The additional intervention of the algorithm in constraining the parameterization of the interaction matrix to positively defined values (where declared) is indicated with the boxes. Other nulled terms correspond to the terms which are nulled by definition of the interaction matrix.

When the interaction matrix is simplified (several terms being nulled as shown in FIGS. 16 and 17), the resulted parameterization of the generalized interaction matrix that can also be used in prediction of TiO₂ nanotubes is presented in FIG. 19B. The additional intervention of the algorithm in constraining the parameterization of the interaction matrix to positively defined values (where declared) is again indicated with the boxes.

### i) Scenario-Dependent Fit to In Vitro Experiments

By parameterized interaction matrix, the fit to the early evolution of the observables for the case of in vitro monitoring of the TiO₂-nanotube exposed lung alveolar model can now be plotted against time for all 6 scenaria. The plots are represented in FIG. 8 (top and middle row correspond to the TiO₂-nanotube exposure related scenaria).

### j) Interaction Mechanism Presentation

By using graphical pictograms (each pictogram refers to the selected observable) defined in FIG. 9, the mechanism, by which observables interact between themselves in case of TiO₂-nanotube exposed lung alveolar model, is schematically depicted in FIG. 10.

Presentation of the mechanisms is based on the parameterized interaction matrix (see results in Step h) using block organization and the concepts explained before-here briefly summarized:
Effects are depicted by arrows with triple encoding (transparency, color and thickness)—those almost transparent, gray and thin are insignificant
Quantities that are affected lie in the inner circle, while the quantities that cause the effect lie on the outer circle
Self-stimulating and self-inhibitory effects are depicted with semi-circular arrows

### k) Dose Biodistribution Characterization

In this example, toxicant biodistribution is determined via image analysis as a local dose (here in unit of toxicant area using ToC0, ToC1 and ToC2 observables per cell area using C1, C2).

10 ROI images from the lower z-slice are used to determine the histogram of dose distribution.

Local 3D stack (on one ROI) is used to calibrate transformation from surface to volume concentrations.

In this example, surface-to-surface dose is used in calculation, which is comprehended by the definition of all compartments (Ci—cell surfaces, ToCi—toxicant surfaces).

In this example, mass-to-surface (of lungs) dose is shown.

In this example, cell-related image channel delivers intensity (density) of labelled membranes. Because it is labelling-dependent it must be uncoupled from labeling efficiency and all experimental factors to deliver the required information on local membrane surface. This is translated with the following consideration:
The required membrane surface per pixel dS/dNpx can be rewritten as the inverse product of labelling efficiency dl/dS and actually measured intensity histogram

$$dNpx / dI \\ dS / dNpx = 1 / ( dI / dS dNpx / dI )$$

It can be shown experimentally, that the labelling efficiency function changes slowly with intensity because of the repartitioning of the labels in the systems (they always try to dilute them locally), for this reason this function might be approximated as a constant; nevertheless, this can be calibrated experimentally;
The inverse of the intensity histograms used by the equation above thus represents the increase of the surface with increased intensity and defines the transformation from intensity to surface; it reflects the fact that at small intensities surface actually does not depend on intensity, because of the under-labelling, while, on the other hand, at higher intensities surface would strongly increase; because the over-labelling is experimentally always avoided, the latter thus reflect only the dense packing of the local membranes (such in case of lipid bodies and local endoplasmic reticulum stacks);
The above consideration is used to empirically fit the inverse intensity histogram by translated potential function with exponents larger than 2 (cut-off for lower intensities to avoid using noisy pixels), and use this fit to translate intensity into surface by means of the equation above with calibrated slope throughout the experimentally derived average distance between packed membranes in cells.

The material related dose is calculated from toxicant-related image channel. Because it is a back-scattered image, the intensity here can be approximated to be proportional to the surface of toxicant (material).

Local dose distribution is finally calculated as the ratio between local surface of material and local membrane surface pixel-wise.

An example, how dose histogram is calculated is shown in the FIG. 12.

### l) Dose-Dependent Long-Time Propagation of Observables

For the chronic-inflammation prediction related to inhalation of TiO₂ nanotubes, as in this Example, the interaction matrix is finally expanded by introducing:
one local-to-systemic coupling (one new row, Ps1) depicting leaking chemokine 3 out from in vitro model (Pl2) into blood stream (there are already 3 local terms related to Chemokine 3, 12 and their interference within in vitro model); because the leak depends on the formation of the gaps between or within epithelial cell, which are in turn related to toxicant area inside epithelial cells (ToC1), the final form of the local-to-systemic term is coupling term is

$$\frac{d}{dt} {Ps}_{1} = pL 2 {S}_{1} F ( {Pl}_{2} , {ToC}_{1} )$$

one systemic-to-local coupling (one new term in C2 differential equation), that describes the PS1-induced migration of the monocytes from blood into lung alveolae and the associated differentiations into macrophages (increasing the local macrophage surface, i.e. C2) (in vivo observations indicate the onset of the systemic response already within 5-15 min); the additional terms looks like

$$\frac{d}{dt} {C}_{2} = … + pS 2 {L}_{1} F ( {Ps}_{1} )$$

Finally, entire expanded interaction matrix is assembled (in vitro parameterized part+in vivo predetermined local-to-systemic and systemic-to-local coupling). The values of additional parameters (here, pL2S₁ and pS2L₁ are taken from intravital in vivo microscopy and from in vivo data where mice were exposed to the TiO₂ nanotubes for validation purposes). With full expanded interaction matrix, the observables are time propagated on much larger time scale (here 800 h-app. 1 month) compared to in vitro monitoring (here for 30 h-app. 1 day) and plotted (FIG. 20, bottom, left and right graph correspond for the smallest and the largest local doses, respectively). Values at the maximum time propagation (here 800 h) of the in-vivo-relevant/predictive observables (in this case the number of macrophages—FIG. 20, bottom—blue lines C2) are plotted for each dose over the dose histogram (FIG. 20, top—blue dots, secondary axes).

This case (exposure to test TiO₂ nanotubes) is used
to prove that the presently disclosed method is able to independently reproduce the same mechanism of relocation (Block R of the interaction matrix, FIG. 10) representing the same cycling of nanomaterial published by H. Kokot et al.; and
to validate the correct values of the local-to-systemic and systemic-to-local coupling from in vivo data (PMN influx measurements at day 1 and 28 and monocyte influx measured at first 4 hours).

### Example 2—Exemplify the Possibilities to Explore and Interpret the Differences in Mechanism of Triggering Chronic Inflammation Between Metal-Oxide Nanotubes (for which Mechanism is Known) and Carbon Nanotubes (for which Mechanism is Unavailable but In Vivo Data is Known for Validation)

After parameterization of the interaction matrix is performed in Step h) for both materials—metal-oxide nanotubes (TiO₂) and carbon nanotubes (MWCNT), mechanism of adverse outcome triggering is shown using mechanism presentation concept from Step j) in FIGS. 10 and 11 for TiO₂ and MWCNT, respectively.

Using the same in vitro system and in vivo-relevant observables as in the Example 1 and disregarding the blocks G and M, which directly describe the in vitro system by itself, the user firstly interprets direct toxicant effects:
Toxicant-modulated growth—block T,
Toxicant relocation—block R,
Toxicant-modulated phenomena—block S.

Block T represents different kind of toxicity of toxicant to all the cell types used within an in vitro model, in this case the effect of two type of nanomaterials with different sizes, chemical composition, surface properties, etc. to the lung alveolar epithelium, including direct toxicity (toxicant inside/uptaken into particular cell type), indirect toxicity (toxicant uptaken into one cell type or compartment and affecting other cell type or compartment) and contact toxicity (toxicant being outside of the cells and affecting particular cell type). Taking a close look on the FIGS. 10 and 11 one can see, that metal-oxide nanotubes are slightly toxic to both cell types when uptaken into macrophages (one direct and one indirect toxic effect). On the other hand, there is no such toxic effect in MWCNT. They rather stimulate growth of some cell than harm them. Block R describes the relocation of the toxicant between the cell types and compartments. Taking a close look on the FIGS. 10 and 11 one can see, that the metal-oxide nanotubes are slowly accumulating in epithelial cells with major part accumulated in quarantine. On the other hand, MWCNTs rather weakly accumulate in quarantine, although there is balance mixture between accessible and quarantine (inaccessible) form of these nanotubes.

Block S shows phenomena, in our case the release of two early cytokines CCl3 (Pl2) and Cxcl12 (Pl1). Normally they are excreted by macrophages (C2) and epithelial cells (C1) as visible from block M. Taking a close look on the FIGS. 10 and 11 one can see that after exposure, free metal-oxide nanotubes (indirectly) as well as those internalized into epithelial cells (directly) stimulate the excretion of Cxcl12, which is normally not excreted. On the contrary, MWCNT suppress the excretion of Cxcl12. In case of CCl3, the situation is much more similar in both cases—note that this cytokine is always excreted under normal conditions.

In the second step, the secondary effects are considered, addressing to:
Phenomena-modulated growth—block C
Phenomena interference—block 2

Block C resembles the effect of cytokines on in vitro model growth. Under normal conditions, they maintain steady cultures. Taking a close look on the FIGS. 10 and 11 one can see that, when exposed to metal-oxide nanotubes, CCl3 seems to suppress both cell types growth and Cxcl12 stimulates both cell type growth. In case of MWCNT, the effect of CCl3-based suppression disappears.

Block 2 represents the 2^{nd} order effects of the cytokines on the release of cytokines. Taking a close look on the FIGS. 10 and 11 one can see that in our case, no difference can be seen between the two exposures.

Finally, the subtlest effects can be shown, if the interaction matrix was allowed to include non-zero blocks B and D. In this case, these effects are:
Phenomena modulation of toxicant accessibility—block B and
Cell modulation of toxicant accessibility—block D

Block B describes changes in accessibility of toxicants because of the action of the cells itself, for example biodegradability and bio-induced aggregation. Taking a close look on the FIGS. 10 and 11 one can see that in our case, exposure to metal-oxide does not associate with any significant changes in accessibility, while exposure to MWCNT slightly shifts accessible (biological effect of the forms) between free form and quarantine.

Block D delivers changes in accessibility of toxicant because of the action of phenomena, in our case due to cytokines. Taking a close look on the FIGS. 10 and 11 one can see that in our case mixed but stronger effects appear in case of MWCNT, which means that the latter interfere with biological signalling (adsorb to or release from the surface of nanotubes).

### Example 3—Exemplify Possibilities in Using Various In Vitro Models in Step b)

The choice of the in vitro model thus first depends on the adverse outcome that might be triggered by the agent as well as on the observables that are related to the in vivo observed events leading to the identified adverse outcome. The in vitro model selection might also depend on tested substance as well, for example inhaled particles are tested on in vitro models relevant for lungs.

The most straightforward approach is simply to construct an in vitro model from cells which constituted the organ, that is affected or related by the adverse outcome, or the organ, that is primarily exposed to the selected agent. But there are also in vitro models that are required to address secondary exposures or more complex situations, that are considered mechanistically plausible.

For example, the inhalation-based exposure logically leads to in vitro models that resembles the function of the lung. These might however comprise various in vitro models, for example in vitro model that mimics the alveolar epithelial layer (air-to-liquid interface, that is exposed to smaller particles entering alveolae—FIG. 3—section Lungs, Epi cells), immune system within the alveolae (FIG. 3—section Lungs, Imu cells), bronchial epithelium (exposed to the larger particles that do not reach alveolae), any combination of these in vitro models (FIG. 3—section Lungs, Epi+Imu) and their further complex with fibroblasts (FIG. 3—section Lungs, Epi+Imu+Fibro) that introduce many other features to the tissue (ability to create excessive amount of extracellular matrix).

Inhalation-based exposures can also affect neural tissues, such as Olfactory barrier, which is suspected to enable direct transport of some substances into the central neural system leading to diseases such as neurodegeneration with extra-high socio-economic impact (FIG. 3—section Brain, Epi), that can logically be expanded with immune cells of the neural tissue-glia cells (FIG. 3—section Brain, Epi+Imu).

One can further enrich the immune system by adding various other immune cells to any of the in vitro models (FIG. 3—section Immune system, dendritic or T-cells).

To address secondary exposures, even with inhalation route as the basic delivery route, the most straightforward in vitro model to address secondary exposure is the vascular endothelial with additional immune system component (monocytes, etc.). Of course, more distant organs can also be affected leading to in vitro models that mimic epithelial layers in kidneys (FIG. 3—section Kidney). Because some of the agents can accumulate in organs, severe long-term health complications can occur, i.e. cancer-some even with huge societal burden leading to in vitro models that address early changes in those systems (FIG. 3—sections Breasts, Umbilical cord, Uterus).

### Example 4—Exemplify Various Possibilities in Acquisition Process of Step c)

The choice of the methods to be used in acquisition process depends on the type of toxicant as well as on the type of observables identified in Step a). Any combination of methods selected to perform Step c) must enable time-dependent detection of all the desired specific events in an in vitro model selected within Step b).

FIG. 4 shows an (non-exclusive) exemplary list of such possible methods (discussed below). These methods are always combined with respect to the type of observables used in acquisition process.

For the observables related to cell properties (identity, shape, surface, dimensions, structure, function, etc.) the following methods are contemplated:
fluorescence microscopy (in confocal or wide-filed version) using specific fluorescent labeling to localize specific cell types or specific targets inside the cells;
fluorescence microscopy (in confocal or wide-filed version) using partition-based fluorescent labelling to localize all organelles or compartment with the same physico-chemical properties such as plasma membrane, all membranes, cytoplasm, actin cytoskeleton, mitochondria, etc.
brightfield (transmission) optical microscopy, when lower resolution is sufficient,
non-linear microscopies (second- or third-harmonic generation) as non-fluorescent label-free imaging method
super-resolution fluorescent techniques such as Stimulated Emission Depletion (STED) microscopy, when higher resolution is required,
electron (SEM, TEM) and ion microscopies (HIM), when localization need to be performed with even higher resolution (to resolve finer details than 10-30 nm), where, however, more advanced sample holders or chamber conditions control concept need to be used to allow time-lapse acquisition or imaging at physiological conditions.

When observables relate to particular local environment, the following methods might be employed:
lifetime fluorescence imaging (FLIM)
hyperspectral imaging (microspectroscopy)
   but all require advanced image decomposition techniques.

When observables are related to dynamics of a local biological component or toxicant (diffusion, mobility, migration, etc.), fast time-lapsed techniques are required such as:
Fluorescence Correlation Spectroscopy (FCS),
Fluorescence Cross-Correlation Spectroscopy FCCS,
time-lapsed imaging with AI-based tracking and identification, etc.,
   but all require advanced image/signal analysis.

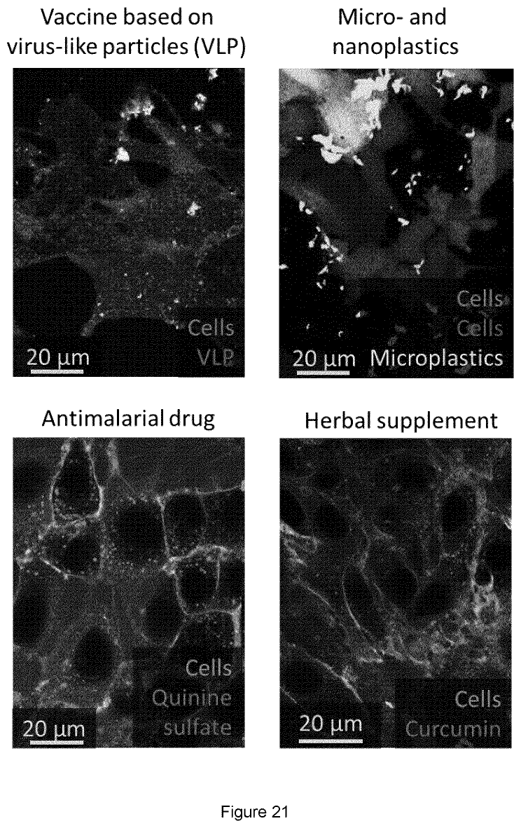

The observables related to tracking of toxicant or local cell compartment/molecules/supramolecular complexes identified via specific chemical properties and related to their concentration, amount, mass, surface, charge, etc., various imaging techniques might be considered:
fluorescence microscopy techniques for any toxicant objects that naturally fluoresce or can be fluorescently labelled ahead of the in vitro exposure experiment (some drugs and supplements, organic and inorganic particles, supramolecular complexes such as delivery vehicles, vehicles for vaccines, viruses, aged plastics, etc.—FIG. 21 shows 4 such systems-vaccine based on virus-like particles, micro- and nano plastics, drug and food supplement),
back-scattering light microscopy for toxicant objects (inorganic particles, some nano plastics, etc.), which are larger than 1/10 of wavelength of light and are locally crystalline or exhibit large electron density or period electron density profiles,
Fourier Transform InfraRed microscopy (FTIR), Raman microscopy (the most sensitive version is Stimulated Raman microscopy), X-ray induced fluorescence (XRF), Secondary-ion mass spectroscopy (SIMS), Proton induced X-ray emission (PIXE) and similar microspectroscopies and hyperspectral microscopies for toxicant that cannot be fluorescently labeled (most of small molecules) or do not fluoresce naturally (many drugs);
electron (SEM, TEM) or ion microscopies (HIM) for all the objects that are at least few nanometers in size, where, however, 3D localization is performed by additional edging or cutting techniques (like microtome TEM, cryo TEM, FIB SEM, etc.).

The observables related to specific (DNA) transcription or (protein) expression, various omics approaches and other specific detection techniques are used:
transcriptomics,
proteomics,
lipidomics,
ELISA.

### Example 5—Exemplify Various Possibilities in Quantification Process of Step d)

FIG. 5 presents an example of 5 possible quantification processes illustrating how the images are processed and observables quantified. In this case, 2 observables are related to cell properties (cell surface of epithelial and immune cells, C1 and C2, respectively) and 3 are related to toxicant surface concentrations in different compartments (toxicant surface in cell type 1—epithelial cells—ToC1, toxicant surface in cell type 2—immune cells—ToC2, and toxicant surface being quarantined, i.e., made inaccessible—ToCQ).

The shown example illustrates the process that is used to quantify an observable from
image-derived mask
z-stack-derived mask
mask derived by multiplication, summation or subtraction of several masks
mask derived by object segmentation and filtering
mask derived by mathematically processing of all the masks above

Here, the first step is used to transform 3D cell locations-z-stack of fluorescence images into proper masks for epithelial cell (C1) and immune cells (C2). Two probes are used to differentiate two cell types—the first probe P1 identifies all the cells (Cs), while the other one P2 depicts the epithelial cells only. The first mask C1 is thus derived as max-projection of the P1 z-image-stack and is directly assigned to the C1 mask. The second mask C2 results as a max projection of P2 z-image-stack from which P1 z-image-stack is subtracted.

In the second step, the back-scattered image BS of the material (toxicant) is first thresholded delivering the total material mask (To). The To mask is segmented into list of identify larger objects (aggregates) of the toxicant. Then, the interface regions (surface) of these aggregates are excluded to derive a mask for quarantined toxicant ToCQ. Because the mask of all non-aggregated material is delivered as the complement of the quarantined material ToCQ mask to total material mask To, the mask of the internalized toxicant quantities in the corresponding cell types, ToC1 and ToC2, respectively, is derived as a product of non-aggregated material mask with particular cell type mask (C1 and C2).

After all masks are created, the amount of toxicant is simply derived by multiplying the toxicant intensity image and the corresponding masks.

1-40. (canceled)

41. A method for predictive testing of agents to predict possibility of agent-associated adverse outcomes based on time propagation of evolution of at least two observables (qᵢ) using a system of differential equations, wherein
an observable (qᵢ) is any property that changes during a set of in vitro experiments, and
each observable (qᵢ) is measured experimentally in vitro at certain time points {tₘ} during at least one of in vitro experiments within the said set of in vitro experiments, and
at least one observable (qᵢ) is causally relatable to known adverse effects, which are detectable in vivo or ex vivo after exposure to the tested agent, which are associable with a key event that unavoidably leads to the next event in a chain of events from exposure to adverse outcome (AO) and
at least one of the in vitro experiments comprises of an exposure of in vitro model to a selected agent, for which prediction is searched for,
characterized in that
said observable (qi) is analyzed, measured and/or detected in a time-lapse manner, i.e., in at least three time points, to obtain measured evolution of each observable (qi), and
in silico-based time-propagation of in vitro detectable observables (qᵢ) is achieved by translating said measured evolution of each observables (qᵢ) into a set of parameterizable interaction terms IMᵢⱼ, which define the system of differential equations suitable for needed time propagation of observables (qᵢ), and
wherein each interaction term IMᵢⱼ is built from predefined classes of interaction functions and defines a change of an observable (qᵢ) due to interaction with at least one another observable (qⱼ), and,
wherein the interaction terms, assembled into an interaction matrix IM(t), define a vector of time derivatives for observables (qi), and
wherein during parametrization of the interaction matrix values of time points, observables and their time derivates at given time points are used as an input

42. The method for predictive testing of agents according to claim 41, wherein the system of differential equations is adjusted to describe the evolution of entire in vitro system or only a part of it by selecting the interaction terms relevant for particular experiment using the concept of scenaria [symbol] via multiplication of the interaction matrix IM(t) by scenaria vector [symbol] wherein a scenario vector is defined as [symbol] ={u₁, u₂, . . . }, where uᵢ are unity values 0 or 1, and where nonzero values of uᵢ are used to select those measurable observables {qᵢ} that are associated with particular experimental setup, which is a combination of in vitro (sub)system and exposure, and wherein an in silico forecasting of at least two in vitro-detectable and in vivo relevant observables qᵢ is achieved by their propagation in time from their initial values [symbol] (t=0) using a system of differential equations defined through the matrix equation [symbol] (t)=IM(t)· [symbol] defined by the interaction terms of an interaction matrix IM(t), selected by the scenaria [symbol] .

43. The method for predictive testing of agents according to claim 41, wherein the interaction matrix term IMᵢⱼ consists of a product of a power parameter pᵢⱼ and at least two interaction functions IF that defines the dependence of the interaction matrix term IMᵢⱼ on the observables qₖ wherein each interaction matrix element is defined as
$${IM}_{i , j} = {p}_{i , j} {\prod }_{k ∈ i , {j}^{*}} {IF}_{o ( k )} ( {q}_{k} )$$
with power parameter p_{i,j} defining the influence of each interaction term to the change rate of the observable qᵢ.

44. The method for predictive testing of agents according to claim 43, wherein for determination of parameters p_{i,j}, a linear combination of linearly independent expressions is used
$$\frac{d}{dt} {q}_{i} = {\sum }_{j} ( {p}_{i , j} {\sum }_{i} ( {\prod }_{k ∈ {i}_{l} , {j}_{l}^{*}} {F}_{o ( k )} ( {q}_{k} ) ) ) = {\sum }_{j} ( {p}_{i , j} {B}_{i , j} ( \{ {q}_{b} \} ) ) \qquad [ 1 ]$$
where B_{i,j} (sum of products of interaction functions) associated with p_{i,j} becomes a base function dependent on a union of observables {q_{b}} that appear in the interaction functions inside the inner sum of Eq. Error! Reference source not found., wherein parametrization with eq. 12 is done in subgroups of equations, in which only a subgroup of parameters pᵢⱼ appears, while other parameters do not—they appear only in the complement of this particular subgroup of equations, and parameterization with eq. 12 is solved with any suitable optimization routine, such as linear or nonlinear optimization routines, preferably with singular value decomposition (SVD), wherein time points tₘ, observables qᵢ(tₘ) and their corresponding derivatives are replaced by their measured values.

45. The method for predictive testing of agents according to claim 41, wherein monitoring of in vitro testing is performed for up to 1 week, preferably up to 3 days, most preferably up to 50 hours, and wherein the observables are acquired at least at three different time points with arbitrary delays and wherein the minimum delay is 5 minutes.

46. The method for predictive testing of agents according to claim 41, wherein the observable is a change of molecular, for example lipid, protein, RNA, DNA, etc., or supramolecular, for example membrane, ribosome, cytoskeleton, fibers, vesicles, etc., or cellular, for example cell surface, volume, shape, activity, cellular compartment surface, volume, shape, etc., property that can be quantified, and wherein the events are selected from the group comprising:
changes in morphology, shape, and mobility of cells,
aggregation and/or dissociation of cells,
altered gene expression,
increased or decreased lipid expression,
increased or decreased transport of molecules and other structures into or from the extracellular space (vesicles, etc),
disconnecting contacts between cells (tight connections),
actin and tubulin rearrangement,
chromatin (de)condensation and other nuclear phase changes,
cell lysis,
apoptosis or necrosis events or events related to other cell state changes,
changes in ER, endosomes, lysosomes, mitochondria and/or ribosomes,
cell death,
quarantining, phagocytotic or other new structure formation on the surface of cells or inside cells, for example quarantine being described by Kokot et al.,
transcription, synthesis, expression, relocation and release of attractants from immune and other type of cells,
binding state, charge, interaction surface chemical composition of molecules, molecular complexes and supramolecular structures, as well as aggregated structure (endogenic or exogenic),
type of interaction exhibited by the interaction surface by any cellular organelle or molecular complex, supramolecular structure or external/exogenic materials,
release and/or absorbance of a molecule, vesicle, etc from one inter/intra-cellular compartment into another,
Surface area of cells of the chosen type (corresponds also to number of cells or concentration of cells, etc.),
Surface area of the toxicant in a chosen compartment (corresponds also to mass, concentration, volume, etc.)
Amount of signalling or transporting molecules or other structures (corresponds also to mass, concentration, binding state, etc. of cytokines, chemokines, enzymes, receptors, RNA of various types, exosomes, endosomes, lipid bodies, etc.),
Surface area of new or modified structures (corresponds also to mass, volume, density, lifetime, dimensionality, correlation lengths, shape descriptors, etc. of supramolecular structures such as vesicular structures, plasma or internal membranes, fibres inside or outside the cells, tight junctions between the cells, gaps between the cells, mitochondria or mitochondrial network, nucleus or parts/domains in nucleus, etc.).

47. The method for predictive testing of agents according to claim 41, wherein the choice of observables depends on the tested substance and pre-existing knowledge on development of possible substance-related outcomes or symptoms, and is based on AOPs, previous in vivo data and/or scientific reports and articles, wherein
The chosen in vitro model is relevant to the particular agent assessment, mimicking at least a relevant part of the relevant target tissue(s) as well as the relevant agent delivery path,
every cell type or their combination used
structurally or functionally mimics AOP-relevant part of the targeted tissue, and
is able to express one or more AOP-relevant key events identified from prior knowledge;
all cell types together are able to respond with a relevant early part of AOP
preceding the later part of the AOP, which are observed in vivo and which develops into observed adverse effect, and
leading to at least one key event that has the power to predict the adverse outcome,
wherein the AOP refers to generalized concept of Adverse Outcome Pathway that comprises all the causally connected events from the Molecular initiating event and all the following events to the final adverse outcome, independently whether the event is physical, chemical, biological, biophysical, biochemical or of any other type.

48. The method for predictive testing of agents according to claim 47, wherein cells for the in vitro model are selected from a group consisting of at least epithelial lung cells, epithelial skin cells, neurons, endothelial cells, muscle cells, intestinal epithelium cells, mucous cells, parietal cells, chief cells, endocrine cells, immune cells, such as macrophages, glia, and similar.

49. The method for predictive testing of agents according to claim 41, wherein quantification of observables in control (no substance) and exposed (tested substance applied in a selected dose) in vitro models depends on the type of the acquired data:
in case the acquired data is already in a numerical form, for example for ELISA, transcriptomics, and/or proteomics, it is directly used for derivative generation,
in case the acquired data is not in a numerical form, for example when images were acquired, the analysis performs a two-step method of:
masking the parts of images that associate with the events-observables, for example type of cell lines, labelling of cells or cellular structures when appropriate, sensitivity of the methods, colocalization information, etc, and
quantification of the corresponding observable, wherein the masks from the previous step are translated into quantified observable q at each time t and each region of interest (ROI), wherein normally, the surface areas are obtained by summing up the number of pixels within the mask and the amounts are derived by summing up the intensities of the appropriate image channel(s) within the mask.

50. The method for predictive testing of agents according to claim 49, wherein quantification results from multiple ROIs are summed up.

51. The method for predictive testing of agents according to claim 41, wherein the already parameterized part of the interaction matrix is expanded to implement additional biological response information on the level of tissue based on AOP or other in vivo data sources representing by local-to-system coupling and system-to-local coupling, wherein
a. the additional part of the interaction matrix provides the mean to couple local events to systemic events and vice versa, and
b. the interaction matrix terms IMᵢⱼ of this expanded part of the interaction matrix can have arbitrary although predefined forms and can depend on any combination of observables from the inside of the in vitro model, as well as of observables from outside the in vitro model, and
c. the parameters pᵢⱼ of interaction matrix terms of this expanded part of the interaction matrix are defined throughout the calibration/validation process by comparing the predicted outcome to the measured outcome from in vivo data sources.
